SEQUENCE LISTING (10 sequences)

SEQ ID NO: 1 - DNA, 26 bp, Artificial Sequence
  synthetic
aggtaccgtcgatcgtgtcggtcgtc

SEQ ID NO: 2 - DNA, 43 bp, Artificial Sequence
  synthetic
tcagagcgcttttgaagctaattcgatctcgcccagctcgatc

SEQ ID NO: 3 - DNA, 43 bp, Artificial Sequence
  synthetic
aggaacttcaagatccccaattcgacaaggactatctcgcgac

SEQ ID NO: 4 - DNA, 25 bp, Artificial Sequence
  synthetic
aggatccgtcgttgttgatcgcgcc

SEQ ID NO: 5 - DNA, 25 bp, Artificial Sequence
  synthetic
cgaattagcttcaaaagcgctctga

SEQ ID NO: 6 - DNA, 24 bp, Artificial Sequence
  synthetic
cgaattggggatcttgaagttcct

SEQ ID NO: 7 - DNA, 19 bp, Artificial Sequence
  synthetic
gcgttcccgaacgtccagc

SEQ ID NO: 8 - DNA, 19 bp, Artificial Sequence
  synthetic
cgtgacggatcacctcgcg

SEQ ID NO: 9 - DNA, 21 bp, Artificial Sequence
  synthetic
cggtgattcagttgcacgtgg

SEQ ID NO: 10 - DNA, 20 bp, Artificial Sequence
  synthetic
gctgcaggtaacggttcggc

(19) United States
(12) Patent Application Publication
(10) Pub. No.: US 2011/0060021 A1
(43) Pub. Date: Mar. 10, 2011

(54) HISTONE DEACETYLASE INHIBITORS AND USES THEREOF

(76) Inventors: Yiqiang Cheng, Grafton, WI (US); Cheng Wang, Whitefish Bay, WI (US)

(21) Appl. No.: 12/859,656

(22) Filed: Aug. 19, 2010

Related U.S. Application Data
(60) Provisional application No. 61/235,253, filed on Aug. 19, 2009.

(51) Int. Cl. A61K 31/4178; C07D 513/02; C12N 9/78; A61K 31/395; C12N 5/09; A61K 31/404; A61P 35/00; A61P 35/02

(52) U.S. Cl. 514/397; 540/456; 435/227; 514/431; 435/375; 514/414

(57) ABSTRACT

Provided herein is a compound of Formula I:

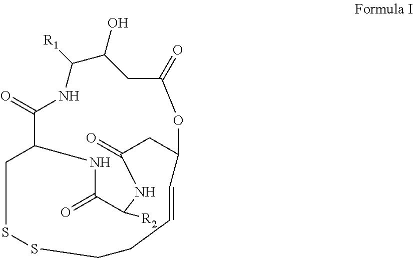

wherein R₁ and R₂ are as disclosed herein, and including reduced forms and dehydration products, and salts thereof. Also provided are compositions, including pharmaceutical compositions, methods of inhibiting histone deacetylase, methods of increasing histone deacetylase-controlled gene expression in a cell, methods of treating a disease associated with increased histone deacetylase activity, methods of inhibiting growth of a cancer cell, and methods of treating cancer in a subject that comprise a compound of Formula I.

### CROSS-REFERENCE TO RELATED APPLICATIONS

This application claims the benefit of priority to U.S. Provisional Patent Application Ser. No. 61/235,253, filed Aug. 19, 2009, which is incorporated by reference herein in its entirety.

### STATEMENT REGARDING FEDERALLY SPONSORED RESEARCH

This work was supported in part by a grant from the U.S. National Institute of Health (R03AI073498), and an Idea Award from the U.S. Department of Defense (DoD) Breast Cancer Research Program (BC073985). The United States government has certain rights in this invention.

### SEQUENCE LISTING

The sequence listing is filed with the application in electronic format only and is incorporated by reference herein. The sequence listing text file “0208719048US01.SeqList.txt” was created on Aug. 19, 2010, and is 2,066 bytes in size.

### BACKGROUND

Histone deacetylase (HDAC) inhibitors are a diverse group of molecules that can induce growth arrest, differentiation, apoptosis, and autophagocytic cell death of cancer cells (Piekarz et al., Clin. Cancer Res. (2009) 15:3918-3926; Yoo et al., Nat Rev Drug Discov., (2006); 5: 37-50). Hence, HDAC inhibitors are prime agents for the development of novel anticancer drugs (Bolden et al., Nat. Rev. Drug Discov. (2006) 5:769-784; Bots et al., Clin. Cancer Res. (2009) 15:3970-3977; Lane et al., J. Clin. Oncol. (2009) 27:5459-5468). One synthetic HDAC inhibitor, vorinostat (suberoylanilide hydroxamic acid—SAHA; commercial name Zolinza® by Merck & Co.), and one natural HDAC inhibitor, FK228 (depsipeptide, romidepsin; commercial name Istodox® by Gloucester Pharma), were recently approved by the U.S. Food and Drug Administration for the treatment of cutaneous T-cell lymphoma (StatBite: FDA oncology drug product approvals in 2009. J. Natl. Cancer Inst. (2010) 102:219; Mann, et al., The Oncologist (2007) 12:1247-1252). Many more HDAC inhibitors (mostly synthetic small molecules) are in various stages of preclinical or clinical trials as single agent or in combination with other chemotherapy drugs for diverse cancer types, including both hematologic and solid tumors (Lane et al., J. Clin. Oncol. (2009) 27:5459-5468; Ma et al., Drugs (2009) 69:1911-1934; Wang et al., Expert opinion on therapeutic patents (2009) 19:1727-1757).

The naturally produced HDAC inhibitor FK228 (C₂₄H₃₆N₄O₆S₂; molecular weight, 540.2) is a bicyclic peptide that was identified in the fermentation broth of Chromobacterium violaceum no. 968 in a screening program that was designed to identify agents that reverse the malignant phenotype of a Ha-ras oncogene-transformed NIH 3T3 cells (Okuhara et al., U.S. Pat. No. 4,977,138; Shigematsu et al., J. Antibiot (Tokyo) (1994) 47:311-314; Ueda et al., J. Antibiot. (Toyko) (1994) 47:315-323; Ueda et al., J. Antibiot (Tokyo) (1994) 47:301-310; Ueda et al., Biosci. Biotechnol. Biochem. (1994) 58:1579-1583).

The number of sequenced microbial genomes has increased dramatically in the past decade (Ng et al., Methods Mol. Biol. (2010) 628: 215-226), and a large number of these microbial genomes often contain many cryptic natural product biosynthetic genes and gene clusters having unknown functions and unidentified biosynthetic products (Cone et al. Nat. Prod. Rep. (2009) 26:977-986; Donadio et al. Nat. Prod. Rep. (2007) 24:1073-1109). Consequently, genome mining has emerged as an effective approach for the discovery of new natural products and development of new compounds associated with genome-sequenced microorganisms (Cone et al. Nat. Prod. Rep. (2009) 26:977-986; Li et al., BMC bioinformatics (2009) 10:185).

Despite the fact that FK228 has been approved by the FDA as a new anticancer drug, dose-limiting toxicities associated with side-effects including fatigue, nausea, vomiting, and anorexia have been observed during clinical trials (Piekarz et al., Curr. Pharm. Des. (2004) 10:2289-2298; Prince et al., Clin. Cancer Res. (2009) 15:3958-3969; Schrump, Clin. Cancer Res. (2009) 15:3947-3957). Accordingly, there remains a need to identify and engineer molecules that inhibit HDAC and subsequently assess the clinical relevance of such molecules in the treatment of conditions and diseases associated with abnormal HDAC activity. Emerging techniques such as genome mining and combinatorial biosynthetic approaches can provide effective tools for identifying and engineering such molecules.

### SUMMARY OF THE INVENTION

In one aspect, the disclosure provides a compound of Formula I:

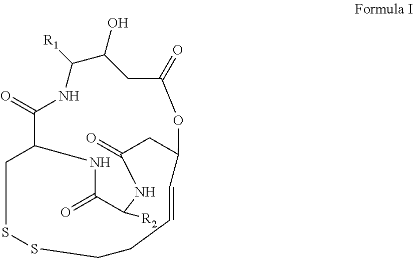

wherein
R₁ and R₂ are independently selected from an amino acid side chain, or derivative thereof, with the proviso that when R₁ is CH(CH₃)₂ or CH(CH₃)CH₂CH₃, R₂ is not CH₃;
   or a reduced form of the compound, a dehydration product of the compound, or a salt thereof.

In an aspect, the disclosure provides a compound of Formula I:

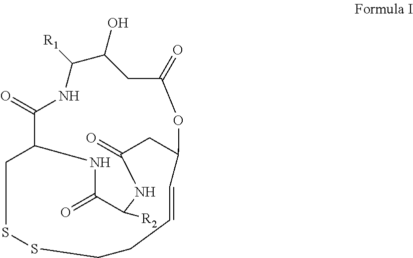

wherein
R₁ is selected from any amino acid side chain, or derivative thereof; and
R₂ is selected from CH₂CH(CH₃)₂, CH(CH₃)CH₂CH₃, (CH₂)₃CH₃, (CH₂)₂SCH₃, (CH₂)₂CONH₂, (CH₂)₃NH₂, (CH₂)₂CO₂, (CH₂)₄NH₃, (CH₂)₃NHC(NH₂)NH₂⁺, (CH₂)₂SeCH₃,

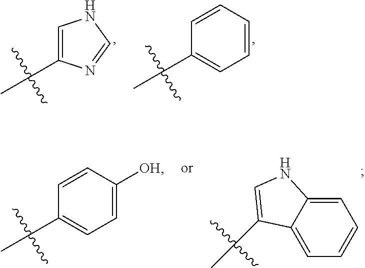

or a reduced form of the compound, a dehydration product of the compound, or a salt thereof.

In an aspect, the disclosure provides a pharmaceutical composition comprising a compound of Formula I:

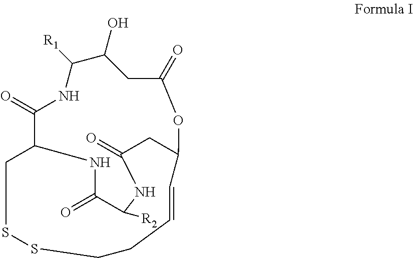

wherein
R₁ and R₂ are independently selected from an amino acid side chain, or derivative thereof, with the proviso that when R₁ is CH(CH₃)₂ or CH(CH₃)CH₂CH₃, R₂ is not CH₃;
or a reduced form of the compound or a dehydration product of the compound; and
a pharmaceutically acceptable carrier or pharmaceutically acceptable salt thereof.

In an aspect, the disclosure provides a pharmaceutical composition comprising a compound of Formula I:

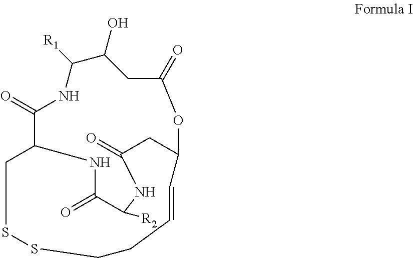

wherein
R₁ is selected from any amino acid side chain, or derivative thereof; and
R₂ is selected from CH₂CH(CH₃)₂, CH(CH₃)CH₂CH₃, (CH₂)₃CH₃, (CH₂)₂SCH₃, (CH₂)₂CONH₂, (CH₂)₃NH₂, (CH₂)₂CO₂, (CH₂)₄NH₃, (CH₂)₃NHC(NH₂)NH₂⁺, (CH₂)₂SeCH₃,

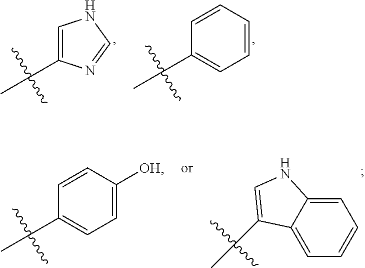

or a reduced form of the compound or a dehydration product of the compound; and
a pharmaceutically acceptable carrier or pharmaceutically acceptable salt thereof.

In another aspect, the disclosure provides a method of inhibiting a histone deacetylase comprising contacting a cell with a compound or salt thereof, or a pharmaceutical composition comprising a compound, of Formula I in an amount effective to inhibit histone deacetylase activity.

In an aspect, the disclosure provides a method of inhibiting histone deacetylase in a subject comprising administering to the subject the compound, a pharmaceutically acceptable salt thereof, or a pharmaceutical composition comprising a compound, of Formula I in an amount effective to inhibit histone deacetylase activity.

In an aspect the disclosure provides a method of increasing histone deacetylase-controlled gene expression in a cell comprising: identifying a gene that has its expression mediated by histone deacetylase activity; and contacting the cell that includes the gene with a compound, or a salt thereof, of Formula I in an amount effective to increase expression of the gene.

In a further aspect, the disclosure provides a method of treating a disease associated with increased histone deacetylase activity in a subject comprising administering a compound, or a pharmaceutically acceptable salt thereof, of Formula I in an amount effective to treat the disease.

In yet another aspect, the disclosure provides a method of inhibiting growth of a cancer cell comprising contacting the cancer cell with an effective amount of a compound of Formula I, or a reduced form or dehydration product, or salt thereof.

In a further aspect, the disclosure provides a method of treating cancer in a subject in need of such treatment, the method comprising administering to the subject an effective amount of a compound of Formula I, or pharmaceutically acceptable salt thereof, or a reduced form or dehydration product of the compound of Formula I.

The disclosure provides for and encompasses additional aspects and embodiments, which will be apparent to those of skill in the art.

### BRIEF DESCRIPTION OF THE DRAWINGS

FIG. 1 depicts the chemical structures of (a) selected histone deacetylase (HDAC) inhibitors, and (b) substrates used in HDAC inhibition assays.

FIG. 2 depicts a comparative map of the FK228 biosynthetic gene cluster (dep) and the thailandepsin biosynthetic gene cluster (tdp). Each gene cluster is depicted in a row with gene names marked above and a deduced biosynthetic pathway drawn under. NRPS, nonribosomal peptide synthetase; PKS, polyketide synthase. A, ACP, AL, AT, C, DH, E, KR, KS, PCP and TE are standard abbreviations of NRPS or PKS domains whose full name and function can be found in (Fischbach & Walsh, Chem. Rev. (2006); 106:3468-3496). Sfp and AT are the generic protein names of their respective genes listed in Table 1.

FIG. 3 depicts a proposed model for the biosynthesis of thailandepsins A and B. The proposed model reflects domain and module organization of six deduced NRPS- and PKS-type enzymes (TdpA, TdpB, TdpC1, TdpDE1, TdpC2 and TdpE2) encoded by the tdp gene cluster and suggests a hybrid NRPS-PKS biosynthetic pathway model which also includes three discrete enzymes (AT, TdpF and TdpH). This proposed pathway contains eight NRPS/PKS modules responsible for seven consecutive steps of building block polymerization that results in a full-length linear intermediate installed to a peptidyl carrier protein (PCP) domain on the last module. A terminal TE domain is predicted to cleave off the intermediate and subsequently cyclize it into a macrolactam intermediate. Finally an FAD-dependent disulfide oxidoreductase (TdpH) is predicted to catalyze a disulfide bond formation as the final step of the biosynthesis of final products.

FIG. 4 depicts the structure and molecular properties of thailandepsins A and B as elucidated by MS and NMR analyses. The proposed structure and molecular properties of thailandepsins C, D, E, and F are also provided.

FIG. 5 Histone deacetylase (HDAC) inhibition assays of compounds (in reduced and oxidized form) against recombinant human HDAC1-4 and HDAC6-9. (A) Bundled HDAC inhibition activity curves for oxidized and reduced FK228. (B) Bundled HDAC inhibition activity curves for oxidized and reduced thailandepsin A. (C) Bundled HDAC inhibition activity curves for oxidized and reduced thailandepsin B. (D) The calculated IC50 value, based on the assay curves, of each non-reduced and reduced (*) compound against each HDAC in μM concentration. TCEP, tris(2-carboxyethyl)phosphine hydrocloride used as a reducing agent.

FIG. 6 shows NCI-60 single-dose (10 μM) assay summarizing inhibitory activity of thailandepsin A against various cancer cell lines.

FIG. 7 shows NCI-60 single-dose (10 μM) assay summarizing inhibitory activity of thailandepsin B against various cancer cell lines.

FIG. 8 shows NCI-60 five-dose assay (100 μM, 10 μM, 1 μM, 0.1 μM, 0.01 μM) summarizing inhibitory activity of thailandepsin A against various cancer cell lines.

FIG. 9 shows NCI-60 five-dose assay (100 μM, 10 μM, 1 μM, 0.1 μM, 0.01 μM) summarizing inhibitory activity of thailandepsin B against various cancer cell lines.

### DETAILED DESCRIPTION

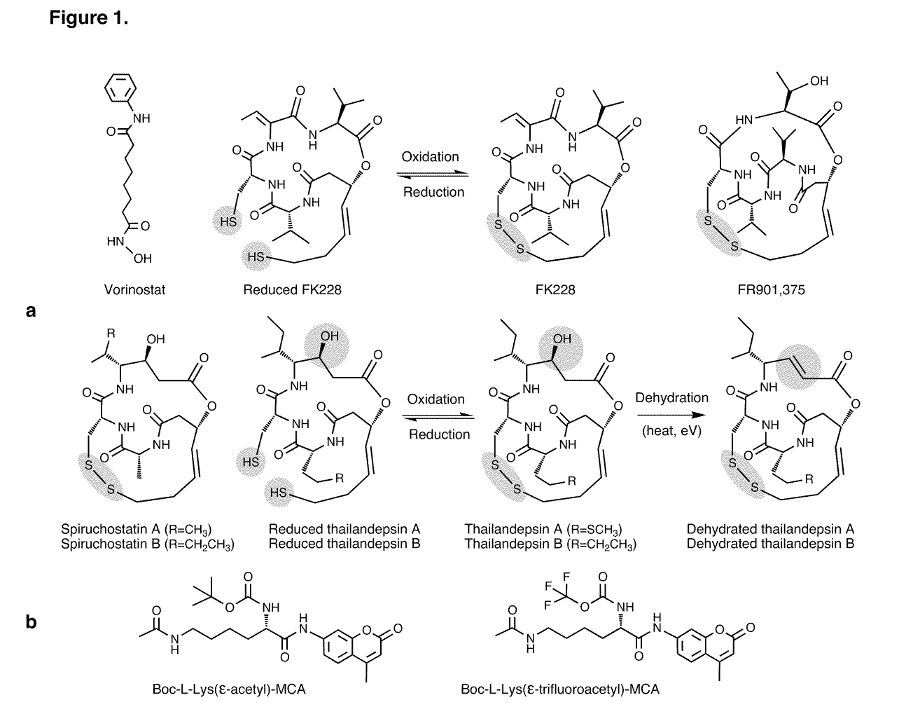

Compounds of Formula I are generally based on a bicyclic peptide structure found in a small family of compounds produced by rare Gram-negative bacterial species. These compounds include thailandepsin A and thailandepsin B (described herein), FK228 (Okuhara et al., U.S. Pat. No. 4,977,138; Shigematsu et al., J. Antibiot (Tokyo) (1994) 47:311-314; Ueda et al., J. Antibiot. (Toyko) (1994) 47:315-323; Ueda et al., J. Antibiot (Tokyo) (1994) 47:301-310; Ueda et al., Biosci. Biotechnol. Biochem. (1994) 58:1579-1583), FR901,375 (Okuhara Masakuni, et al., JP Patent Publication No. 03141296 (abstract)), spiruchostatins A and B (Masuoka et al., Tetrahedron Lett. (2001) 42:41-44) (FIG. 1). Without being limited by any mechanism or implied functionality, these compounds comprise a disulfide bond that is proposed to mediate a mode of anticancer action in which a reduced thiol group “warhead” interacts with a Zn²⁺ ion located in the catalytic active site of Class I and Class II HDACs and inhibit HDAC enzymatic activity (Cheng et al., Appl. Environ. Microbiol. (2007) 73:3460-3469; Furumai et al., Cancer Res. (2002) 62:4916-4921).

The inventors' prior experience in identifying FK228 and elucidating its biosynthetic pathway (Cheng et al., Appl. Environ. Microbiol. (2007) 73:3460-3469; Potharla et al., submitted, “Systematic gene mutation and transcriptional analysis revealing unexpected genetic organization and pathway regulation of the FK228 biosynthetic gene cluster in Chromobacterium violaceum no. 968” (2010) (Potharla et al.); Wesener et al., submitted, “Reconstitution of FK228 biosynthetic pathway revealing cross-talk between modular polyketide synthases and fatty acid synthase (2010) (Wesener et al.)), have identified compounds of Formula I (including thailandepsins A and B) that act as HDAC inhibitors and can be used as anticancer agents.

While the biosynthesis of FK228 has been reported, the following provides a brief summary. The biosynthesis of FK228 is proposed to follow a widely accepted “assembly-line” mechanism (Du et al., Genet. Eng. (NY) (2003) 25:227-267; Finking et al., Annu. Rev. Microbiol. (2004) 58:453-488; Fischbach et al., Chem. Rev., (2006); 106:3468-3496; Walsh, Science (2004) 303:1805-1810), in which simple building blocks (amino acids, amino acid derivatives and short carboxylic acids from primary metabolism) are polymerized step-wise by seven modules of a hybrid nonribosomal peptide synthetase (NRPS)-polyketide synthase (PKS) multifunctional pathway to afford a linear intermediate that is subsequently cyclized by a terminal thioesterase (TE) domain to form an immediate FK228 precursor (Cheng et al., Appl. Environ. Microbiol. (2007) 73:3460-3469). An FAD-dependent pyridine nucleotide-disulfide oxidoreductase, encoded by depH, is responsible for a disulfide bond formation on the precursor as the final step in FK228 biosynthesis (Wang et al., Chem. Biol. (2009) 16:585-593).

As described herein, compounds of Formula I can be produced using this assembly-line mechanism, manipulating the various building block units and modules as necessary depending on the particular amino acid side chains that comprise the R₁ and R₂ groups.

As mentioned previously, genome mining is known in the art and provides an effective informational/computational tool for identifying new molecules and biosynthetic machinery. In brief summary, genome mining begins by searching microbial genome sequences available in public databases for characteristic natural product biosynthetic genes or gene clusters. Once target genes or gene clusters are identified, the sequence of the deduced gene products is analyzed and the putative function of each gene is postulated. Next, the modular organization and substrate specificity of biosynthetic enzymes is dissected and predicted. Finally, a putative biosynthetic pathway is constructed and a structure or a series of structures of potential natural product(s) that could be made by the pathway is proposed. These bioinformatic analyses of natural product biosynthetic gene clusters can provide a good prediction of gene categories and functions, gene cluster boundaries, biosynthetic sequence, and regulatory cascade, all of which can be validated and confirmed by experimental validation (Cheng et al., Appl. Environ. Microbiol. (2007) 73:3460-3469; McAlpine et al., J. Nat. Prod. (2005) 68:493-496; Potharla et al., 2010). Experimental validation can be performed using a variety of known chemical, biochemical, spectroscopic, and biological techniques, such as those detailed herein as illustrative examples and that provide validation of thailandepsin A and thailandepsin B.

Thus, in a general sense, the disclosure provides compounds that can inhibit histone deacetylase (HDAC) activity, as well as methods and compositions comprising the compounds. The compounds that act as HDAC inhibitors encompassed by the disclosure are based on compounds termed “thailandepsins” (TDPs), including thailandepsin A and thailandepsin B, which are derived from the Gram-negative bacillus, Burkholderia thailandensis E264, and which have been structurally characterized, as described herein. The thailandepsin biosynthetic (tdp) gene cluster shows homology to the dep gene cluster which contains the genes responsible for the biosynthesis of FK228, an FDA-approved anticancer drug.

Accordingly, the disclosure provides a compound of Formula I:

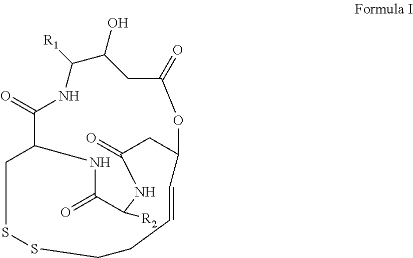

wherein each of R₁ and R₂ are independently selected from an amino acid side chain, a derivative thereof, and including salts thereof. Amino acids are well known to those of skill in the art and are molecules containing an amine group, a carboxylic acid group and a side chain that varies between different amino acids. Amino acids include alpha-amino acids of the general formula H₂NCHRCOOH, where R is an amino acid side chain comprising an organic substituent, as well as uniquely structured amino acids such as, for example, proline. Amino acids include, for example, isoleucine, leucine, alanine, asparagine, glutamine, lysine, aspartic acid, glutamic acid, methionine, cysteine, phenylalanine, threonine, tryptophan, glycine, valine, proline, serine, tyrosine, arginine, histidine, norleucine, ornithine, taurine, selenocysteine, selenomethionine, lanthionine, 2-aminoisobutyric acid, dehydroalanine, hypusine, citrulline, 3-aminopropanoic acid, gamma-aminobutryic acid, and the like. Accordingly, the term “amino acid side chain” refers to the various organic substituent groups (e.g., “R” in H₂NCHRCOOH) that differentiate one amino acid from another. A “derivative” of an amino acid side chain refers to an amino acid side chain that has been modified structurally (e.g., through chemical reaction to form new species, covalent linkage to another molecule, etc.). In embodiments, R₁ and R₂ each comprise a side chain of an amino acid independently selected from any of the twenty common amino acids as well as uncommon amino acids, or derivatives thereof. In embodiments, R₁ comprises the side chain of any amino acid or a derivative thereof, and R₂ comprises CH₂CH(CH₃)₂, CH(CH₃)CH₂CH₃, (CH₂)₃CH₃, (CH₂)₂SCH₃, (CH₂)₂CONH₂, (CH₂)₃NH₂, (CH₂)₂CO₂, (CH₂)₄NH₃, (CH₂)₃NHC(NH₂)NH₂⁺, (CH₂)₂SeCH₃,

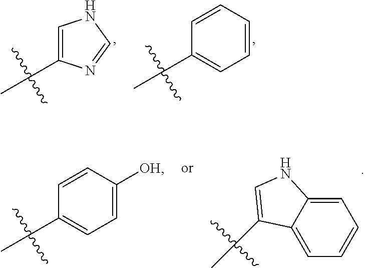

In some embodiments, the amino acid side chain comprising R₂ can be selected to interact with a secondary binding site on an HDAC. The catalytic domains of various HDACs have been identified and characterized, and thus are known in the art. As a non-limiting example, HDACs of class 1 and class 2 comprise a catalytic domain formed by a stretch of about 390 amino acids that include a set of conserved amino acids. Generally, the active site forms a tubular pocket with a narrow opening and wider bottom (See, e.g., Finnin et al., Nature (London) (1999) 401:188-193; de Ruijter et al., Biochem. Journal (2003) 370:737-749; and Annemieke, et al., Biochem. J. (2003) 370:737-749). HDAC activity (i.e., acetyl group removal) is driven by a charge-relay system that includes a Zn²⁺ ion, two histidine residues, two aspartic residues (about 30 amino acids from the adjacent histidines), and a tyrosine residue (about 120 amino acids downstream of the aspartic residues). The secondary binding site is typically located near active site, accessible at the surface of the HDAC. Thus, in further embodiments, a secondary binding site can be identified on an HDAC, typically near the surface of the HDAC in a region close to the active site, and R₂ can be selected based on its ability to interact with the secondary binding site. The extent of binding interaction between R₂ and the secondary binding site of an HDAC can be determined by any method known in the art, such as molecular modeling, binding affinity assays, or photoaffinity labeling (He et al., J Med. Chem. (2009) 52:7003-7013). In such embodiments, R₂ can comprise a larger amino acid side chain (e.g., comprising aromatic and/or longer alkyl moieties). These embodiments also allow for the identification and design of compounds of Formula I that are selective for a particular HDAC, or the HDACs of a particular class (i.e., isoform-selective HDAC inhibitors, see, Balasubramanian et al., Cancer Lett. (2009) 280:211-221). Such HDAC selective compounds of Formula I can provide for reduced adverse effects that might occur through non-specific (or less specific) HDAC inhibition. Methods for such identification of isoform-selective HDAC inhibitors have been described (See, e.g., He et al., J Med. Chem. (2009) 52:7003-7013, incorporated by reference herein). In some embodiments, R₁ and R₂ are independently selected from the group consisting of CH₃, CH(CH₃)₂, CH₂SH, CH₂CH(CH₃)₂, CH(CH₃)CH₂CH₃, (CH₂)₃CH₃, (CH₂)₂SCH₃ and CH(OH)CH₃, with the proviso that when R₁ is CH(CH₃)₂ or CH(CH₃)CH₂CH₃, R₂ is not CH₃. In some embodiments, R₁ is CH₃(CH)CH₂CH₃, and R₂ is CH(CH₃)₂, CH₂SH, CH₂CH(CH₃)₂, CH(CH₃)CH₂CH₃, (CH₂)₃CH₃, (CH₂)₂SCH₃, or CH(OH)CH₃. In some embodiments, R₁ is CH₃(CH)CH₂CH₃, and R₂ is (CH₂)₃CH₃ or (CH₂)₂SCH₃.

In embodiments, the compounds of Formula I can contain the disulfide bond as depicted in Formula I, or the disulfide bond can be reduced, such as the non-limiting examples depicted FIG. 1 (e.g., thailandepsin A and thailandepsin B). Embodiments also provide for dehydration products of the molecules of Formula I, such as the non-limiting examples provided in FIG. 1 (e.g., dehydrated thailandepsin A and dehydrated thailandepsin B). Embodiments also provide for compounds of Formula I as optically pure isomers. The compound of Formula I as well as the reduced and dehydrated compounds of Formula I can also be provided as salts such as, for example, basic or acidic addition salts. The selection and formation of such salts are within the ability of one skilled in the art. See, e.g., Remington: The Science and Practice of Pharmacy, 21^{st} ed., Lippincott Williams & Wilkins, A Wolters Kluwer Company, Philadelphia, Pa (2005).

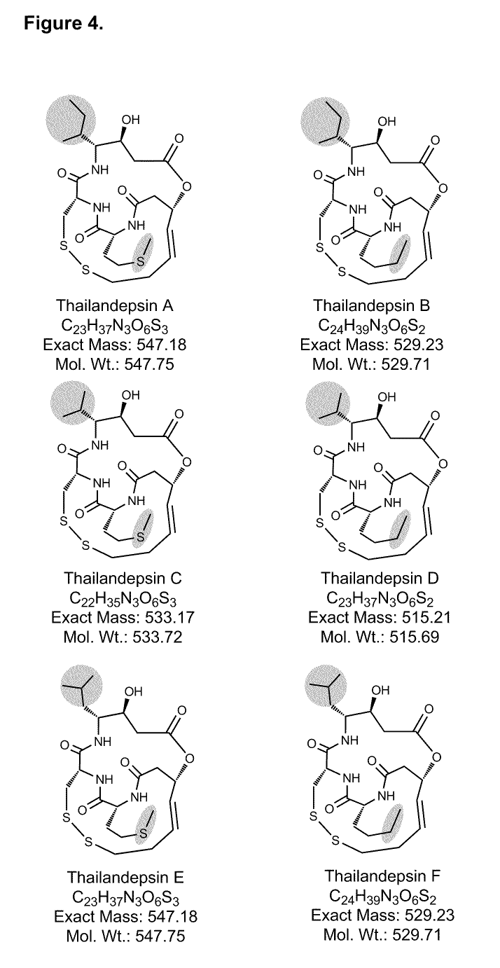

Further non-limiting examples of compounds within the scope of Formula I include, those compounds shown in FIG. 4 and designated as thailandepsins C, D, E, and F as well as, the reduced forms, dehydration products, and salts thereof. Also encompassed are combinations of compounds of Formula I, reduced forms of the compounds of Formula I, dehydration products of the compounds of Formula I, or salts thereof. In some embodiments, the disclosure provides for an isolated and purified compound of Formula I. In further embodiments, the isolated and purified compound of Formula I comprises a thailandepsin such as, for example, a thailandepsin selected from thailandepsins A, B, C, D, E, and F. In yet further embodiments, the compound of Formula I is thailandepsin A or thailandepsin B. Embodiments also provide for a composition consisting essentially of a compound of Formula I. In further embodiments, the composition consists essentially of a thailandepsin such as, for example, a thailandepsin selected from thailandepsins A, B, C, D, E, and F. In yet further embodiments, the composition consists essentially of thailandepsin A or thailandepsin B.

In embodiments described herein, the compounds of Formula I, including thailandepsins, are active inhibitors of histone deacetylase activity. As used herein, “histone deacetylase” or “HDAC” means any eukaryotic protein that can function to remove acetyl groups from an ε-N-acetyl lysine amino acid on a histone (see, e.g., Allis et al., Cell (2007) 131:633-636; Bernstein et al., Cell (2007) 128:669-681). The term is also interchangeable with the terms “lysine deacetylases” and “KDAC.” The compounds of Formula I that function as HDAC inhibitors, as provided herein, can have activity against any of the four known classes of HDACs: Class I, (HDAC 1, 2, 3, and 8); Class IIa, (HDAC 4, 5, 7, and 9), Class IIb (HDAC6 and 10); Class III, (Sir2 in S. cerevisiae, the “sirtuins” SIRT1, SIRT2, SIRT3, SIRT4, SIRT5, SIRT6, SIRT7 in mammals); and Class IV, (HDAC11) (Bolden et al., Nat Rev Drug Discov. (2006) 5:769-784). In embodiments, the compounds of Formula I have inhibitor activity against a mammalian HDAC. In further embodiments, the mammalian HDAC is a human HDAC. In some embodiments the HDAC is a recombinant HDAC.

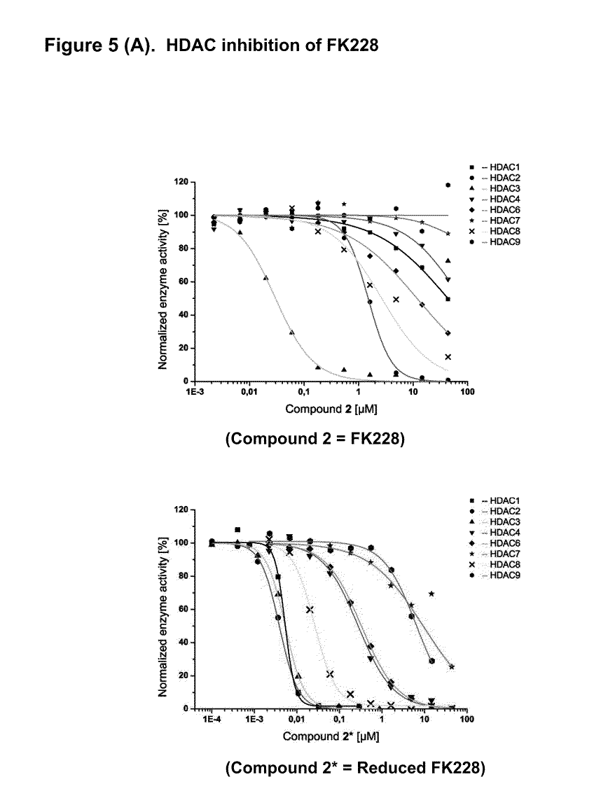

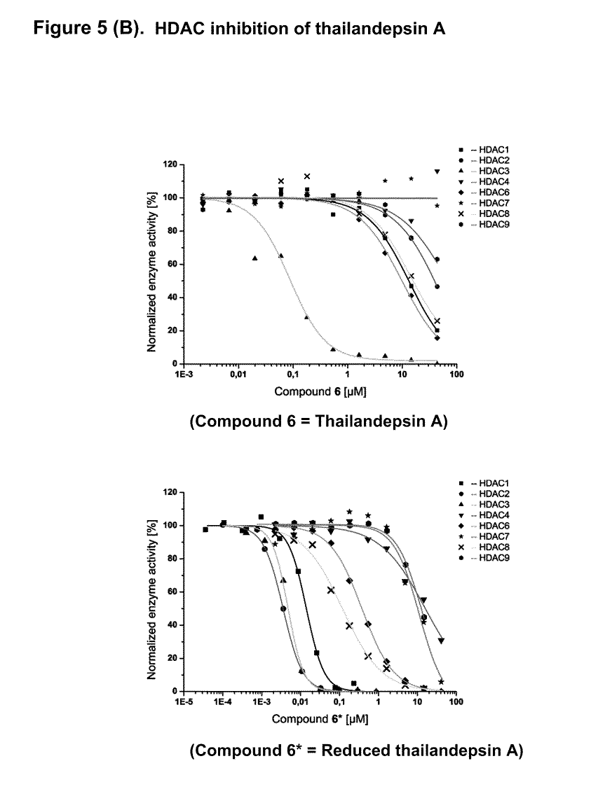

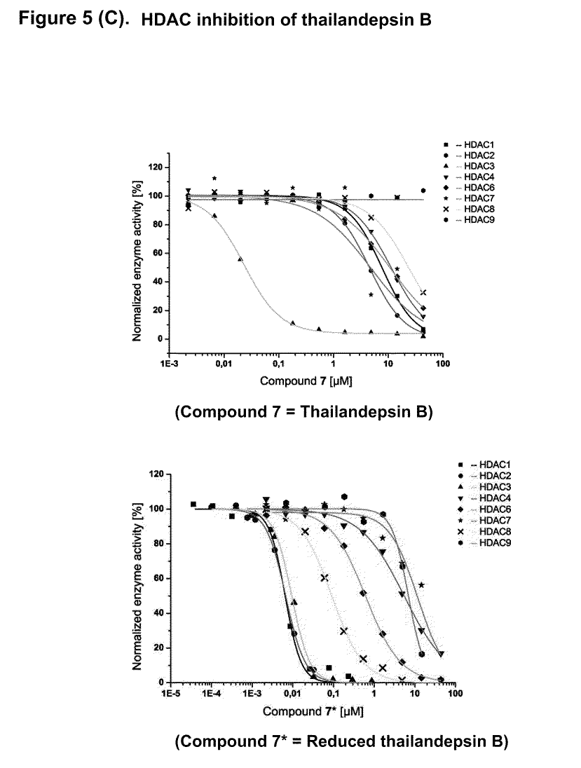

In embodiments, the compounds of Formula I are active inhibitors of HDAC with IC50 values from about 0.1 nM to about 500 μM, from about 0.1 nM to about 100 μM, from about 1 nM to about 50 μM, or from about 1 nM to about 10 μM. The Examples describe a non-limiting type of histone deacetylase inhibition assay (Wegener et al., Analytical Biochemistry (2003) 321: 202-208) that compares the histone deacetylase inhibition activities of thailandepsin A and thailandepsin B to that of FK228 (FIG. 1). Both thailandepsins exhibited histone deacetylase inhibitor activity in a range similar to that of FK228, except against HDAC4, to which thailandepsins have much higher IC50 values (FIG. 5). Any suitable assay for histone deacetylase inhibition, including assays known to those of skill in the art (Wegener et al. Analytical Biochemistry, (2003) 321:202-208) can be used to determine the histone deacetylase inhibitory activity of the compounds of Formula I.

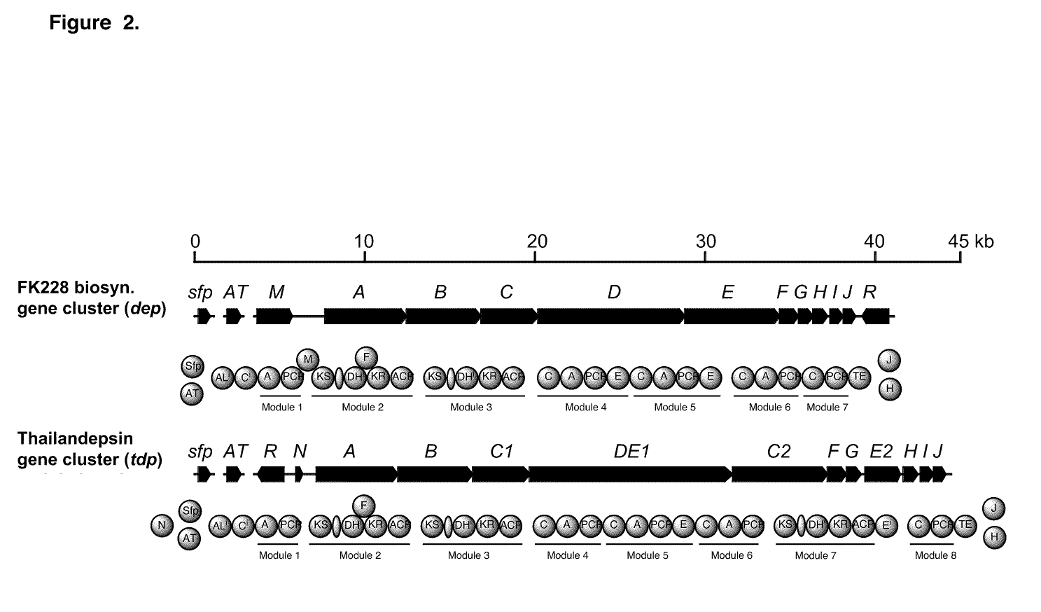

Compounds of Formula I can be synthesized using any technique that is familiar to one of skill in the art. Some embodiments relate to a gene cluster that encodes proteins that are able to generate compounds of Formula I via biosynthesis. For example, the tdp gene cluster that encodes proteins involved in the biosynthesis of thailandepsins has been identified in the genome of Burkholderia thailandensis E264 as described in the Examples (Table 1, FIGS. 2 & 3, and U.S. patent application Ser. No. 12/526,202; PCT/US2008/053473 (WO 2008/098199), each of which is incorporated herein by reference). The sequences of the tdp gene cluster are also available in GenBank (GenBank accession no. CP000085 and CP000086).

TABLE 1
Comparison of the Deduced Proteins of Thailandepsin Biosynthetic (tdp) Gene
Cluster with the FK228 Biosynthetic (dep) Gene Cluster.

| Thailandepsin | | FK228 | | | |
|---|---|---|---|---|---|
| biosynthetic | | biosynthetic | | | |
| (tdp) gene cluster | | (dep) gene cluster [1] | | % Ident./simil. | Confirmed or deduced |
| Gene^{a} | Protein^{b} | Gene^{b} | Protein^{b} | btw prot. seq. | protein function^{c} |
| (Multiple | Bth264_Sfp | Cv968_sfp | Cv968_Sfp | — | Phosphopantetheinyl |
| candidate genes) | | (discrete)^{d} | | | transferase (PPTase) |
| (discrete)^{d} | | | | | |
| (Multiple | Bth264_AT | Cv968_fabD1 | Cv968_fabD1 | — | Acyltransferase, malonyl |
| candidate genes) | | Cv968_FabD2 | Cv968_FabD2 | | CoA-specific (AT) |
| (discrete)^{d} | | (discrete)^{d} | | | |
| BTH_I2369 | TdpR | — | — | — | AraC-type transcriptional |
| | | | | | regulator |
| — | — | depM | DepM | — | Aminotransferase |
| BTH_I2368 | TdpN | — | — | — | Type II peptidyl carrier |
| | | | | | protein (PCP) |
| BTH_I2367 | TdpA | depA | DepA | 74%/83% | NRPS (1 module) |
| BTH_I2366 | TdpB | depB | DepB | 78%/86% | PKS (1 module) |
| BTH_I2365 | TdpC1 | depC | DepC | 76%/84% | PKS (1 module) |
| BTH_I2364 | TdpDE1 | | | | |
| | 1st module | depD | DepD | 56%/67% | NRPS (1 module) |
| | 2nd module | depE | DepE | 39%/52% | NRPS (1 module) |
| BTH_I2363 | TdpC2 | depC | DepC | 39%/50% | PKS (1 module) |
| BTH_I2362 | TdpF | depF | DepF | 88%/93% | FadE2-like acyl-CoA |
| | | | | | dehydrogenase |
| BTH_I2361 | TdpG | depG | DepG | 75%/84% | Phosphotransferase |
| BTH_I2360 | TdpE2 | depE | DepE | 31%/49% | NRPS (partial module) |
| BTH_I2359 | TdpH | depH | DepH | 72%/84% | FAD-dependent disulfide |
| | | | | | oxidoreductase |
| BTH_I2358 | TdpI | depI | DepI | 74%/84% | Esterase/Lipase |
| BTH_I2357 | TdpJ | depJ | DepJ | 67%/80% | Type II thioesterase |
| — | — | depR | DepR | — | LysR-type transcriptional |
| | | | | | regulator |

^{a}Gene annotations from the GenBank;
^{b}Gene/protein names designated herein;
^{c}Standard abbreviations: NRPS, nonribosomal peptide synthetase; PKS, polyketide synthase;
^{d}Detached from the perspective gene cluster;
—: not available.
[1] Reference Cheng Y. Q., et al., Appl. Environ. Microbiol. (2007) 73: 3460-3469; Potharla et al., 2010; Wesener et al., 2010.

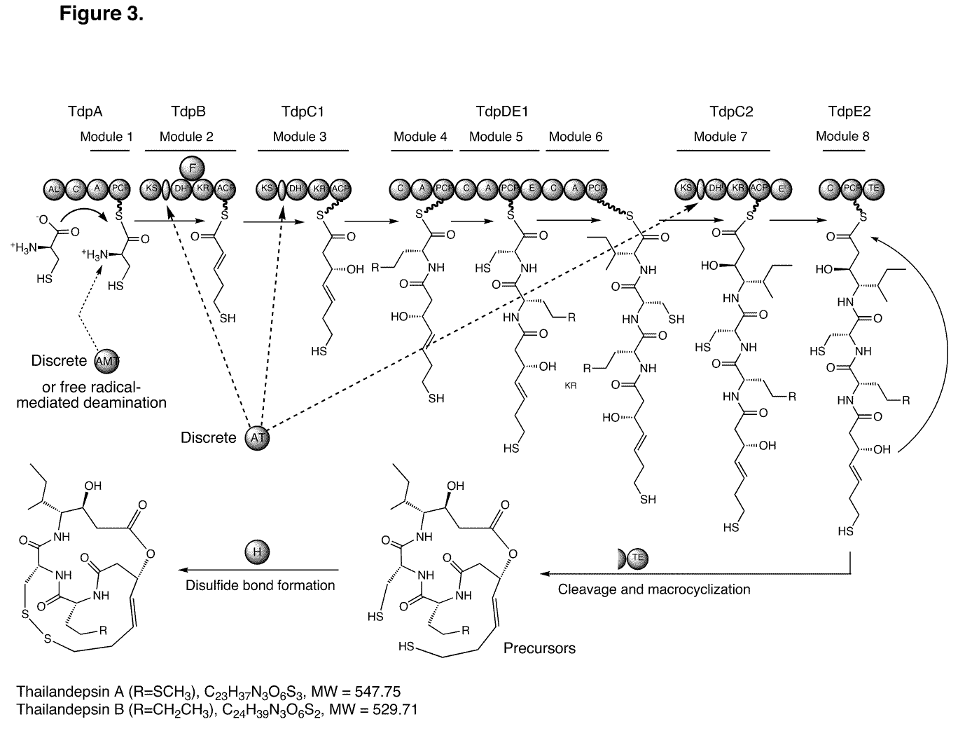

Accordingly, compounds of Formula I can be biosynthesized by a scheme similar to the proposed synthetic scheme for thailandepsin, as depicted in FIG. 3. For purposes of illustration, methods of producing thailandepsins by growing Burkholderia thailandensis E264 are provided herein. Burkholderia thailandensis E264 can be grown in medium under conditions that allow for production of the thailandepsins, and can be adapted from media and growth conditions such as are known in the art, as well as those as described in the Examples. Thailandepsins can then be recovered and isolated from the growth medium. Those of skill in the art can appreciate that the compounds of Formula I can also be isolated using a variety of common isolation techniques, including but not limited to, organic extraction, column chromatography, and HPLC. Preliminary mass spectroscopy analysis indicates that several thailandepsins, designated thailandepsins C to F, are also likely biosynthesized by Burkholderia thailandensis E264 (see, FIG. 4). The structures of thailandepsin A and B have been confirmed by infra-red spectroscopy, mass spectrometry, and extensive 1D and 2D NMR spectroscopy, as noted in the Examples. Thailandepsin A and B are also described in the Examples as a non-limiting illustration that the compounds of Formula I act as histone deacetylase inhibitors (FIG. 5). Due to the expected structural similarity, the compounds of Formula I, along with the other compounds of the thailandepsin type, are expected to inhibit histone deacetylases and have anti-cancer properties.

### Methods

In aspects, the compounds of Formula (I) can be used to treat diseases associated with abnormal histone deacetylase activity, which includes increased histone deacetylase activity, by administering an effective amount of at least one compound of Formula (I) to a subject with such a disease. Diseases characterized by abnormal histone deacetylase activity include, but are not limited to, inflammatory disorders, diabetes, diabetic complication, homozygous thalassemia, cystic fibrosis, cirrhosis, various cancers including acute myeloid leukemia (AML), chronic lymphocytic leukemia (CLL), neurodegenerative disease, cognitive disorder, and autoimmune disease. (See, Hutt et al., Nat Chem. Biol. (January 2010) 6:25-33; Mai et al., Mol. Pharmacol., (2007) 72(5):1111-23; Mai et al., Curr Pharm Des. (2009) 15(34):3940-57; Rotili et al., Curr Top Med. Chem. (2009) 9(3):272-91; Kazantsev and Thompson, Nat Rev Drug Discov. (2008) 7(10):854-68; Halili et al. Curr Top Med. Chem. (2009) 9(3):309-19, each of which is incorporated herein by reference). The terms “treating” and “treatment” when used with reference to a disease or a subject in need of treatment includes, but is not limited to, halting or slowing of disease progression, remission of disease, prophylaxis of symptoms, reduction in disease and/or symptom severity, or reduction in disease length as compared to an untreated subject. In embodiments, the methods of treatment can abate one or more clinical indications of the particular disease being treated. Certain embodiments relating to methods of treating a disease or condition associated HDAC activity comprise administration of therapeutically effective amounts of a compound of Formula I, as well as combinations of two or more compounds of Formula I, as well as pharmaceutical compositions thereof.

Aspects of the disclosure provide a method of inhibiting histone deacetylase in a cell, including a cell within a subject, comprising contacting the cell with a compound of Formula I in an amount effective to inhibit histone deacetylase activity. In embodiments, the method provides for inhibiting HDAC activity in a cell in a subject, wherein the method includes administering to the subject the compound, or a pharmaceutically acceptable salt thereof, according to Formula I in an amount effective to inhibit histone deacetylase activity in the cell in the subject. Histone deacetylase activity can be monitored by any method familiar to those of skill in the art. In some embodiments histone deacetylase activity can be monitored by clinical evaluation of the symptoms or stage of a disease associated with abnormal histone deacetylase activity.

In some embodiments that relate to a method for inhibiting histone deacetylase activity in a cell, the method comprises increasing histone deacetylase-controlled gene expression in the cell. Accordingly, in further embodiments, the method includes identifying a gene that has its expression controlled by histone deacetylase; and contacting the cell that includes the gene with the compound of Formula I, or a salt thereof, in an amount effective to increase expression of the gene. In embodiments, these methods include contacting the cell with an effective amount of a composition comprising a thailandepsin compound such as, for example, thailandepsin A and/or thailandepsin B, which are capable of inhibiting HDACs as demonstrated in the Examples. The HDACs are known to inhibit gene expression, thus inhibition of HDACs can result in increased expression of genes. In these embodiments, “inhibiting” or “inhibition” of HDAC means that there is a measurable decrease in the activity of HDAC in the presence of a compound of Formula I (e.g., through contacting/administration), relative to the activity of HDAC without the compound of Formula I. In embodiments, HDAC is inhibited by a compound of Formula I by about 10% to about 100% (e.g., about 10%, 20%, 30%, 40%, 50%, 60%, 70%, 80%, 90%, or 100%) relative to a control. In some embodiments compounds of Formula I can inhibit HDAC (e.g., IC50) at concentrations from about 0.1 nM to about 500 μM, (e.g., about 0.1 nM to about 250 μM, about 0.5 nM to about 200 μM, about 1.0 nM to about 100 μM, about 10 nM to about 50 μM, or about 100 nM to about 10 μM, and the like). As used herein, “gene expression” encompasses expression of polynucleotides encoding for polypeptides natively associated with the cell as well as expression of polynucleotides encoding non-native polypeptides. Effects of contacting a cell with a compound of Formula I can be evaluated by comparing expression of a polynucleotide in a cell treated with a compound of Formula I to expression in an untreated cell. Expression of a polynucleotide may be assessed by any means known to those of skill in the art, including but not limited to, RT-PCR, Northern analysis, and Western analysis.

As previously noted, aspects of the disclosure relate to a method of inhibiting growth of a cancer cell comprising contacting the cancer cell with an effective amount of a compound, or salt thereof, of Formula I, as described above. In embodiments, the method comprises treating cancer in a subject in need of such treatment, the method comprising administering to the subject a therapeutically effective amount of a compound, or pharmaceutically acceptable salt thereof, of Formula I. In some embodiments the therapeutically effective amount is an amount sufficient to inhibit the proliferation of the cancer. In some embodiments the therapeutically effective amount is an amount sufficient to slow the progression of the cancer. In further embodiments, the therapeutically effective amount is an amount sufficient to reduce the number of cancer cells in the subject (i.e., killing of cancer cells). Methods for monitoring the proliferation of cancer cells and progress of cancer in a subject (e.g., tumor size, cell counts, biochemical markers, secondary indications, etc.) are known in the art.

In various embodiments of the method, the cancer is associated with the activity of one or more HDACs. Non-limiting examples of cancer that are associated with one or more HDACs include carcinoma, adenoma, melanoma, sarcoma, lymphoma, myeloid leukemia, lymphatic leukemia, blastoma, glioma, astrocytoma, mesothelioma, and or a germ cell tumor. In embodiments, the cancer is from the colon, rectum, cervix, skin, eye, epithelium, muscle, kidney/renal, liver/hepatocellular, lymph, bone, blood/hematopoeitic, ovary, prostate, lung, brain/central nervous system, stomach, gastrointestinal, bladder, endometrial/uterine, thyroid, testicle, or breast. In further embodiments, the cancer is ovarian cancer, renal cancer, colon cancer, melanoma, brain/central nervous system cancer, or breast cancer.

In some embodiments, the method of treatment includes administration of a therapeutically effective amount of a compound of Formula I in combination with an additional anti-cancer agent. A wide variety of anti-cancer (i.e., anti-neoplastic) agents are known in the art and include, for example alkylating agents, antimetabolites, natural antineoplastic agents, hormonal antineoplastic agents, angiogenesis inhibitors, differentiating reagents, RNA inhibitors, antibodies or immunotherapeutic agents, gene therapy agents, small molecule enzymatic inhibitors, biological response modifiers, and anti-metastatic agents.

In some embodiments, the method of treatment can be used an adjuvant therapy (i.e., additional treatment) such as when compounds of Formula I, or pharmaceutical compositions thereof, are administered after surgery or other treatments (e.g., radiation, hormone therapy, or chemotherapy). Accordingly, the methods of treatment described herein encompass those that include administering the compounds of Formula I to a subject either alone or in combination with one or more other treatment regimes. Such other treatments can include surgery, radiation therapy, systemic therapy (e.g., chemotherapy, immunotherapy, hormone therapy, or biological response modifiers). Those of skill in the art will be able to use statistical evidence to assess the risk of disease relapse before deciding on the specific adjuvant therapy. The aim of adjuvant treatment is to improve disease-specific and overall survival. Because the treatment is essentially for a risk, rather than for provable disease, it is accepted that a proportion of patients who receive adjuvant therapy will already have been effectively treated, or cured by their primary surgery. Adjuvant therapy are often given following surgery for many types of cancer including, for example, colon cancer, lung cancer, pancreatic cancer, breast cancer, prostate cancer, and some gynecological cancers.

Some embodiments of the method are related to neoadjuvant therapy, which is given before the main treatment. Effective neoadjuvant therapy is commonly characterized by a reduction in the number of cancer cells (e.g., size of the tumor) so as to facilitate more effective surgery.

In embodiments, the method of treating can relate to any method that prevents further progression of the disease and/or symptoms, slows or reduces the further progression of the disease and/or symptoms, or reverses the disease and/or clinical symptoms associated with a disease associated with increased histone deacetylation.

In embodiments, the methods are used to treat cancer in a subject, wherein the subject is a mammal. Yet further embodiments relate to methods wherein the mammal is a human.

The term “cancer” refers to or describes the physiological condition in mammals that is typically characterized by unregulated cell growth. Some non-limiting examples of cancer include carcinoma, melanoma, lymphoma, blastoma, sarcoma, germ cell tumors, and leukemia or lymphoid malignancies. Non-limiting examples of cancers that fall within these broad categories include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, melanoma, multiple myeloma and B-cell lymphoma, brain, as well as head and neck cancer, and associated metastases.

The term “cancer” also encompasses cell proliferative disorders which are associated with some degree of abnormal cell proliferation, and includes tumors. “Tumor” as used herein, refers to any neoplasm or neoplastic cell growth and proliferation, whether malignant or benign, and all pre-cancerous and cancerous cells and tissues.

Administration of an effective amount of a compound of Formula I, such as a thailandepsin for example, to a subject may be carried out by any means known in the art including, but not limited to intraperitoneal, intravenous, intramuscular, subcutaneous, or transcutaneous injection or oral, nasopharyngeal or transmucosal absorption. Such administration encompasses the administration of a compound of Formula I formulated as a pharmaceutical composition. Delivery (administration route) also includes targeted delivery wherein the compound of Formula I is only active in a targeted region of the body (for example, in cancerous tissues), as well as sustained release formulations in which the compound of Formula I is released over a period of time in a controlled manner. Sustained release formulations and methods for targeted delivery are known in the art and include, for example, use of liposomes, drug loaded biodegradable microspheres, drug-polymer conjugates, drug-specific binding agent conjugates and the like. Pharmaceutically acceptable carriers are well known to those of skill in the art. Determination of particular pharmaceutical formulations and therapeutically effective amounts and dosing regimen for a given treatment is within the ability of one of skill in the art taking into consideration, for example, patient age, weight, sex, ethnicity, organ (e.g., liver and kidney) function, the extent of desired treatment, the stage and severity of the disease and associated symptoms, and the tolerance of the patient for the treatment.

It will be understood that any numerical value recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between the lowest value and the highest value enumerated are to be considered to be expressly stated in this application.

Also, it is to be understood that the phraseology and terminology used herein is for the purpose of description and should not be regarded as limiting. The use herein of terms such as “comprising,” “including,” “having,” and variations thereof is meant to encompass the items listed thereafter and equivalents thereof as well as additional items. “Comprising” encompasses the terms “consisting of” and “consisting essentially of.” The use of “consisting essentially of” means that the composition or method may include additional ingredients and/or steps, but only if the additional ingredients and/or steps do not materially alter the basic and novel characteristics of the claimed composition or method.

All patents publications and references cited herein are hereby fully incorporated by reference.

While the following examples provide further detailed description of certain embodiments of the invention, they are merely illustrative and do not limit the claimed invention.

### EXAMPLES

Bacterial strains and plasmids. Burkholderia thailandensis E264 (ATCC 700388; Am^{R} Km^{R} Gm^{R} Sm^{R} Pm^{R} Tc^{S}; see also, GenBank accession no. CP000085 and CP000086), a gram-negative motile rod bacterial species isolated from a rice paddy in Thailand (Brett et al., 1998), and E. coli were routinely cultured various growth media, including a modified minimal broth [Handbook of Microbiological Media, 2^{nd} Ed. (1997) R. M. Atlas, Editor, Boca Raton, Fla.; CRC Press (pg. 948)] or Luria-Bertani (LB) broth or on agar plates at 37° C. For the construction of a targeted gene-disruption mutant, a suicide vector, pEX18Tc (Tc^{R} oriT⁺ sacB⁺, conjugative), originally developed for Pseudomonas aeruginosa genetics (Hoang et al., Gene (1998) 212:77-86), and a selective marker donor plasmid pBS854-Tp (constructed in house), were used successfully in B. thailandensis.

### Example 1

### Identification of the tdp Biosynthetic Gene Cluster

A proposed biosynthetic gene cluster (designated tdp for thailandepsins) was identified in a review of the published genome of Burkholderia thailandensis E264. The genes and the deduced proteins of this tdp gene cluster exhibit a significant overall similarity to those of the dep gene cluster which is involved in the biosynthesis of FK228 (Table 1; FIG. 2). In particular, the deduced products of eight genes (tdpA, tdpB, tdpC1, tdpF, tdpG, tdpH, tdpI, and tdpJ) share 67%-80% or more sequence identity-similarity with their respective counterpart from the FK228 biosynthetic pathway. Like the dep gene cluster, this tdp gene cluster does not contain any gene that encodes a phosphopantetheinyltransferase (PPTase) necessary for posttranslational modification of carrier proteins (Lambalot et al., Chem. Biol. (1996) 3:923-936) or an acyltransferase (AT) necessary for in trans complementing the three “AT-less” modules of PKS on TdpB, TdpC1 and TdpC2 proteins (Cheng et al., Proc Natl Acad Sci USA 2003 100:3149-3154; Cheng et al., Methods in Enzymology (2009) 459:165-486). A thorough search of the draft B. thailandensis genome identified multiple candidate genes that may encode the missing PPTase or AT activity but unambiguous identification of the responsible genes (tentatively named Bth264_sfp and Bth264 AT genes; Table 1) requires experimental verification. A few exceptions/differences between the two parallel gene clusters are identified as follows: (i) unlike depR which is located downstream of the dep gene cluster and encodes a putative LysR-type transcriptional regulator, tdpR is located upstream of the tdp gene cluster and encodes a putative AraC-type transcriptional regulator. These two deduced regulatory proteins do not have significant homology; (ii) there is no depM counterpart in the tdp gene cluster; (iii) there are two copies of depC-like genes in the tdp gene cluster, the second copy is fused with DNA encoding a likely inactive epimerase (E) domain and is located after tdpDE1; (iv) a depE-like gene in the tdp gene cluster is split into two parts, the first part is fused to the end of tdpD, and the second part is transposed to a downstream location between tdpG and tdpH; (v) unlike pseudogene “depN”, the deduced protein of tdpN appears to be a functional peptidyl carrier protein with a critical serine residue for phosphopantetheinylation.

### Example 2

### Construction of a Targeted Gene-Disruption Mutant of B. Thailandensis

General DNA manipulations, including plasmid preparation, restriction enzyme digestion, agarose gel electrophoresis, and bacterial transformation, were performed according to standard protocols (Sambrook and Russell (2000) Molecular Cloning: A laboratory manual. Third edition. Cold Spring Harbor, N.Y.: Cold Spring Harbor Laboratory.) or the manufacturer's instructions (New England Biolabs). Genomic DNA of the wild type or mutant strain of B. thailandensis was prepared from an overnight culture with an UltraClean microbial DNA isolation kit (MO BIO Labs). A 375-bp DNA fragment toward the 3′-end of tdpA (Amplicon 1) was amplified from B. thailandensis genomic DNA with the following PCR primer set: KpnI-tdpAB-UpF, 5′-AGGTACCgtcgatcgtgtcggtcgtc-3′, (SEQ ID NO:1) containing a KpnI site (underlined); and FRT-F-tdpAB-UpR, 5′-TCAGAGCGCT TTTGAAGCTAATTCGatctcgcccagctcgatc-3′ (SEQ ID NO:2). A 395-bp DNA fragment toward the 5′-end of tdpB (Amplicon 2) was amplified from B. thailandensis genomic DNA with the following PCR primer set: FRT-R-tdpAB-DnF: 5′-AGGAACTTCAAGATCCCCAATTCGacaaggactatctcgcgac-3′, (SEQ ID NO:3) and BamHI-tdpAB-DnR, 5′-AGGATCCgtcgttgttgatcgcgcc-3′ (SEQ ID NO:4) containing a BamHI site (underlined). A 700-bp FRT cassette containing a Tp^{r} marker gene flanked by two FRT recognition sequences (Amplicon 3) was amplified from pPS854-Tp with the following PCR primer set: FRT-F, 5′-CGAATTAGCTTCAAAAGCGCTCTGA-3′, (SEQ ID NO:5) and FRT-R, 5′-CGAATTGGGGATCTTGAAGTTCCT-3′ (SEQ ID NO:6). Amplicons 1-3 were assembled into a 1.45-kb Amplicon 4 by multiplex PCR using Long Amp DNA polymerase (New England Biolabs). Amplicon 4 was digested with KpnI/BamHI and the insert was subsequently cloned into suicide vector pEX10Tc to make a tdpAB-gene replacement vector pYC05-57.

To create a tdpAB-gene replacement mutant of B. thailandensis, pYC05-57 was first transformed into E. coli S17-1 cells which subsequently passed the vector to B. thailandensis cells via interspecies conjugation. Mutant strains of B. thailandensis with tdpAB partially replaced by the FRT cassette were selected on LB agar supplemented with 50 μg/ml apramycin (Am; B. thailandensis is naturally resistant to Am up to 200 μg/ml), 100 μg/ml trimethoprim (Tp) and 5% (w/v) sucrose at 30° C. The genotype of independent mutants was verified by colony PCR using the following primer sets: tdpAB-PCR-FP1 (5′-GCGTTCCCGAACGTCCAGC-3′, (SEQ ID NO:7)) and tdpAB-PCR-RP1 (5′-CGTGACGGATCACCTCGCG-3′, (SEQ ID NO:8)), and tdpAB-PCR-FP2 (5′-CGGTGATTCAGTTGCACGTGG-3′, (SEQ ID NO:9)) and tdpAB-PCR-RP2 (5′-GCTGCAGGTAACGGTTCGGC-3′, (SEQ ID NO:10)). One representative mutant strain was saved and named BthΔtdpAB::FRT.

To create a marker-free mutant by removing the FRT cassette from BthΔtdpAB::FRT, a broad host-range Flp-expression vector pBMTL3-FLP2 (Wang et al., Chem. Biol. (2009); 16: 585-593) was introduced into BthΔtdpAB::FRT by conjugation and the marker-free mutants were selected on LB agar supplemented with 50 μg/ml Am and 25 μg/ml chloramphenicol (Cm) at 37° C. Vector pBMTL3-FLP2 was subsequently cured from the mutants by streaking for two rounds on LB agar supplemented with 50 μg/ml Am and 5% (w/v) sucrose at 30° C. The genotype of independent gene-deletion mutants was verified by colony PCR. One final representative marker-free mutant strain was saved and named BthΔtdpAB.

### Example 3

### Examination of the Metabolic Differences Between the Wild Type (BthWT) and BthΔtdpAB Mutant Strain of B. thailandensis

Since the tdpAB genes were thought to be involved in the biosynthesis of thailandepsins (FIG. 1), partial deletion of tdpAB would abolish the production of thailandepsins in the mutant strain. Detection of the metabolic profile differences between wild type and the BthΔtdpAB mutant strain of B. thailandensis facilitated the identification and purification of thailandepsins.

Organic extracts of mutant and wild type cultures, along with a medium control, were analyzed by high performance liquid chromatography (HPLC) and detected the disappearance of several HPLC peaks in the BthΔtdpAB sample. The corresponding peaks in the BthWT sample were targeted as likely compounds produced by the tdp gene cluster. Those peaks were collected and examined by electrospray ionization-mass spectrometry (ESI-MS). Peak 1 from HPLC yielded a pair of ion signals of 547.9/530.0 m/z, and Peak 2 yielded 530.0/511.9 m/z. It is believed that the higher m/z signal from each pair is the protonated adduct of a target molecule [M+H]⁺ and the lower m/z signal is the protonated adduct of a respective target molecule with a H₂O molecule removed (dehydrated; [M-H₂O+H]⁺) by heat/electrovoltage (eV) during ESI-MS. Interestingly, when the samples were reduced with 50 mM DTT and subjected to ESI-MS again, both generated ion signals with a +2 m/z mass shift, a polarity shift (more hydrophilic, as judged by an earlier elution time), and a change of relative abundance of intact molecule/dehydrated derivative. Together those observations led to the conclusion that the tdp gene cluster in B. thailandensis E264 can generate at least two compounds that likely contain a reducible disulfide bond.

### Example 4

### Purification and Characterization of Thailandepsins

Wild type B. thailandensis E264 strain was fermented in a series of growth media, including (a) a modified nutrient broth (1.0% glucose, 1.0% Difco nutrient broth, 0.5% NaCl, 0.1% CaCO₃, pH 7.0); (b) a modified YM-254890 medium (2.0% glycerol, 0.5% glucose, 0.5% peptone, 0.1% yeast extract, 0.1% NaCl, pH 7.0); and (c) a modified minimal broth “M9” (1.0% glucose, 0.7% K₂HPO₄, 0.2% KH₂PO₄, 0.1% (NH₄)₂SO₄, 0.05% sodium citrate, 0.01% MgSO₄.7H₂O, pH 7.0) at 30′C for 3 days with shaking (200 rpm). Sterile resins, HP-20 and XAD-16 (Sigma-Aldrich, St. Louis, Mo.), for absorbing secreted metabolites, were added to culture to a final concentration of 1.0% (w/v) each at day 2. Additional experiments to determine optimal culture conditions led to the selection of M9 as the best medium for downstream fermentation for 72 h at 30° C. with constant agitation (200 rpm). Resins and cells were collected at the end of fermentation by centrifugation and subsequently freeze-dried for 3 days. The dry mass was extracted five times, each with two volumes of ethyl acetate (w/v). Organic extracts from two fermentation media were combined at this point and the solvent was removed under reduced pressure to give a crude extract. The crude extract was fractionated sequentially by silica gel chromatography, Sephadex LH20 chromoatography, and preparative HPLC. Fractions containing target compounds were identified by LC-MS. Thailandepsins A and B (see, FIG. 1) were obtained as amorphous white powder, and were structurally characterized by chemical derivation, infra-red (IR) and mass spectrometry (MS) analyses, and extensive 1D and 2D NMR analyses.

### Example 5

### Histone Deacetylase Inhibition Assays

The HDAC inhibition activity of the isolated thailandepsins A and B were evaluated relative to the known HDAC inhibitor, FK228, against eight recombinant human histone deacetylases with calculated IC50 values (FIG. 5). The experiments included HDACs falling in human class I (HDACs 1, 2, 3 and 8), as well as human class II (HDACs 4, 6, 7 and 9), and the activities were determined in a two-step fluorogenic assay by the method described in (Wegener et al. Analytical biochemistry (2003) 321:202-208). All HDAC dose-response curves were bundled for each compound individually in the native/oxidized and the reduced forms. The activity of each enzyme was normalized separately. For determination of the inhibitory effect, IC50 values (in μM concentrations) were obtained through a non-linear four-parameter fit. The reduced forms of each of the compounds demonstrated increased inhibitory effect (lower IC50 value) relative to the corresponding non-reduced form of the compound. The reduced form of thailandepsin A inhibits HDAC2≧HDAC3>HDAC1>HDAC8>HDAC6>HDAC7≧HDAC9≧HDAC4 in the order. A slightly different inhibition profile was observed with the reduced form of thailandepsin B as HDAC2≧HDAC1≧HDAC3≧HDAC8>HDAC6>HDAC4≧HDAC9>HDAC7. The reduced form of FK228 exhibited an inhibition ranking as HDAC2≧HDAC3≧HDAC1>HDAC8>HDAC4≧HDAC6>HDAC9≧HDAC7. The data suggests that reduced thailandepsins A and B have a similar HDAC inhibition profile with each other, and which is distinctive from that of reduced FK228, particularly toward inhibitory activity on HDAC4.

### Example 6

### Anticancer Activity of Thailandepsin A and B

Thailandepsins A and B were submitted to the NIH/NCI Developmental Therapeutics Program (DTP) for testing against the NCI-60 cancer cell lines (see, Holbeck et al., Mol. Cancer. Ther. (2010) 9: 1451-1460; Shoemaker, Nat. Rev. Cancer, (2006) 6:813-823; and Monga & Sausville, Leukemia (2002) 16:520-526), and were assigned reference numbers NSC D-751510 and NSC D-751511, respectively, in the NCI database. The NCI-60 DTP Human Tumor Cell Line Screen utilizes 60 different human tumor cell lines, representing leukemia, melanoma and cancers of the lung, colon, brain, ovary, breast, prostate, and kidney. The aim is to prioritize for further evaluation, synthetic compounds or natural product samples showing selective growth inhibition or cell killing of particular tumor cell lines. This screen is unique in that the complexity of a 60 cell line dose response produced by a given compound results in a biological response pattern which can be utilized in pattern recognition algorithms (such as the DTP's COMPARE program). Using these algorithms, it is possible to assign a putative mechanism of action to a test compound, or to determine that the response pattern is unique and not similar to that of any of the standard prototype compounds included in the NCI database. Details regarding the NCI-60 developmental therapeutics program Human Tumor Cell Line Screen can be found on the NCI/NIH developmental therapeutics program website; however an overview of the screening protocols as described on the NCI/NIH DTP website are provided below.

Methodology Of The In Vitro Cancer Screen

The human tumor cell lines of the cancer screening panel are grown in RPMI 1640 medium containing 5% fetal bovine serum and 2 mM L-glutamine. For a typical screening experiment, cells are inoculated into 96 well microtiter plates in 100 μL at plating densities ranging from 5,000 to 40,000 cells/well depending on the doubling time of individual cell lines. After cell inoculation, the microtiter plates are incubated at 37° C., 5% CO₂, 95% air and 100% relative humidity for 24 h prior to addition of experimental drugs.

After 24 h, two plates of each cell line are fixed in situ with TCA, to represent a measurement of the cell population for each cell line at the time of drug addition (Tz). Experimental drugs are solubilized in dimethyl sulfoxide at 400-fold the desired final maximum test concentration and stored frozen prior to use. At the time of drug addition, an aliquot of frozen concentrate is thawed and diluted to twice the desired final maximum test concentration with complete medium containing 50 μg/ml gentamicin. Additional four, 10-fold or ½ log serial dilutions are made to provide a total of five drug concentrations plus control. Aliquots of 100 μl of these different drug dilutions are added to the appropriate microtiter wells already containing 100 μl of medium, resulting in the required final drug concentrations.

Following drug addition, the plates are incubated for an additional 48 h at 37° C., 5% CO₂, 95% air, and 100% relative humidity. For adherent cells, the assay is terminated by the addition of cold TCA. Cells are fixed in situ by the gentle addition of 50 μl of cold 50% (w/v) TCA (final concentration, 10% TCA) and incubated for 60 minutes at 4° C. The supernatant is discarded, and the plates are washed five times with tap water and air dried. Sulforhodamine B (SRB) solution (100 μl) at 0.4% (w/v) in 1% acetic acid is added to each well, and plates are incubated for 10 minutes at room temperature. After staining, unbound dye is removed by washing five times with 1% acetic acid and the plates are air dried. Bound stain is subsequently solubilized with 10 mM trizma base, and the absorbance is read on an automated plate reader at a wavelength of 515 nm. For suspension cells, the methodology is the same except that the assay is terminated by fixing settled cells at the bottom of the wells by gently adding 50 μl of 80% TCA (final concentration, 16% TCA). Using the seven absorbance measurements [time zero, (Tz), control growth, (C), and test growth in the presence of drug at the five concentration levels (Ti)], the percentage growth is calculated at each of the drug concentrations levels.

Percentage growth inhibition is calculated as:

[(Ti−Tz)/(C−Tz)]×100 for concentrations for which Ti≧Tz

[(Ti−Tz)/Tz]×100 for concentrations for which Ti<Tz.

Three dose response parameters are calculated for each experimental agent. Growth inhibition of 50% (GI50) is calculated from [(Ti−Tz)/(C−Tz)]×100=50, which is the drug concentration resulting in a 50% reduction in the net protein increase (as measured by SRB staining) in control cells during the drug incubation. The drug concentration resulting in total growth inhibition (TGI) is calculated from Ti=Tz. The LC50 (concentration of drug resulting in a 50% reduction in the measured protein at the end of the drug treatment as compared to that at the beginning) indicating a net loss of cells following treatment is calculated from [(Ti−Tz)/Tz]×100=−50. Values are calculated for each of these three parameters if the level of activity is reached; however, if the effect is not reached or is exceeded, the value for that parameter is expressed as greater or less than the maximum or minimum concentration tested.

Initial NCI-60 Screen Single-Dose Assay for Thailandepsins A and B

All compounds submitted to the NCI 60 Cell screen, or compounds currently in the screening queue, are tested initially at a single high dose (10⁻⁵ M, or 10 μM) in the full NCI-60 cell panel. Only compounds which satisfy pre-determined threshold inhibition criteria will progress to the 5-dose screen. The threshold inhibition criteria for progression to the 5-dose screen were designed to efficiently capture compounds with anti-proliferative activity and are based on careful analysis of historical DTP screening data. The data is reported as a mean graph of the percent growth of treated cells.

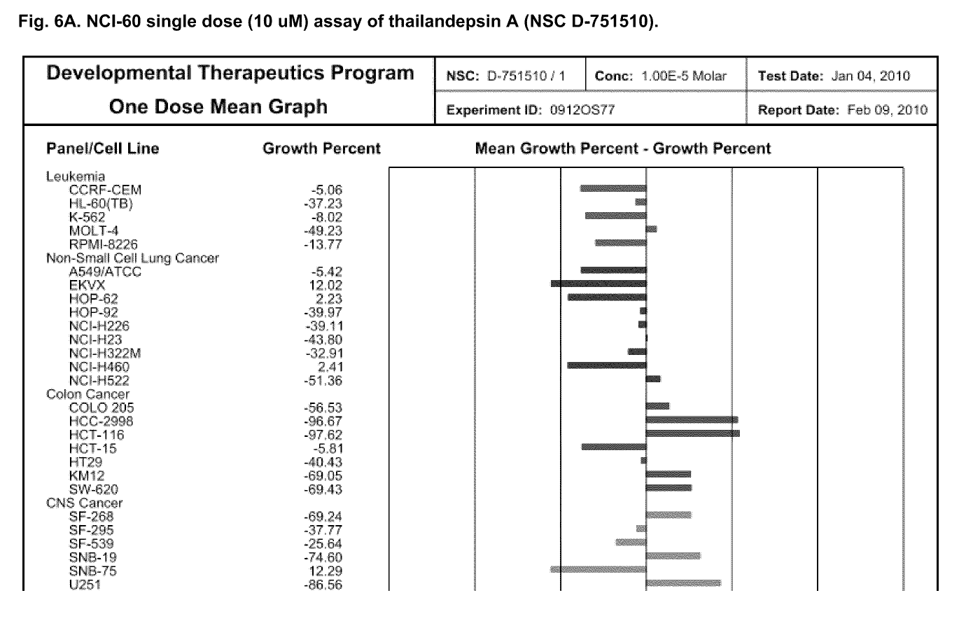

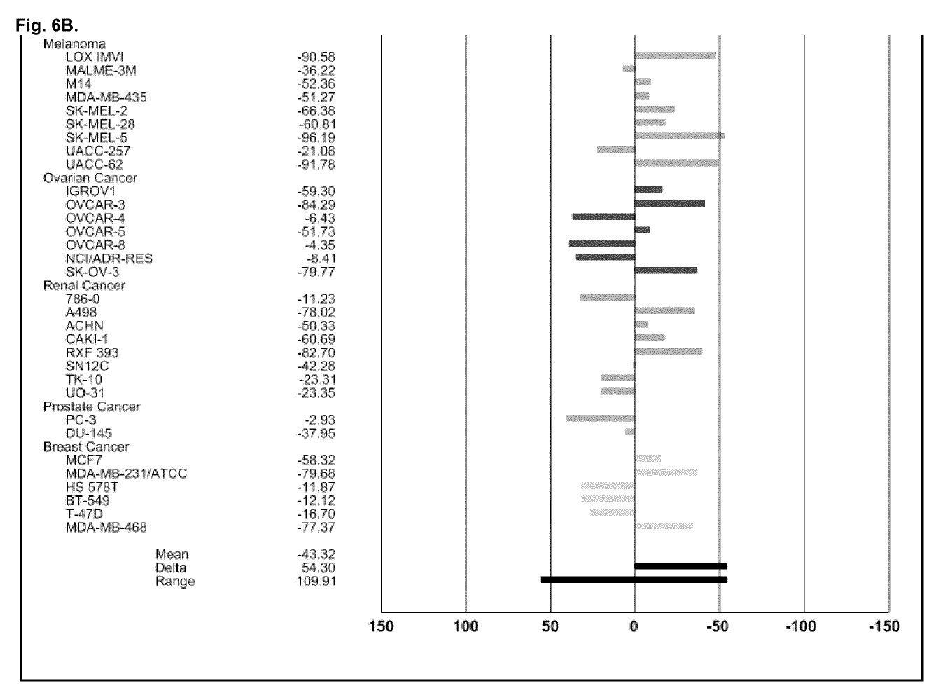

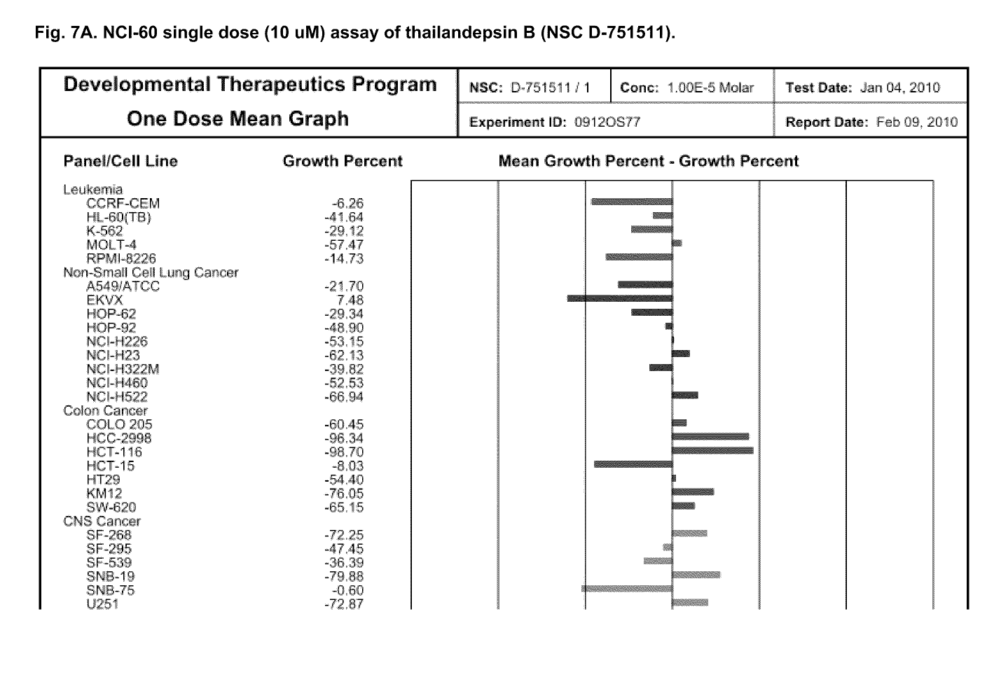

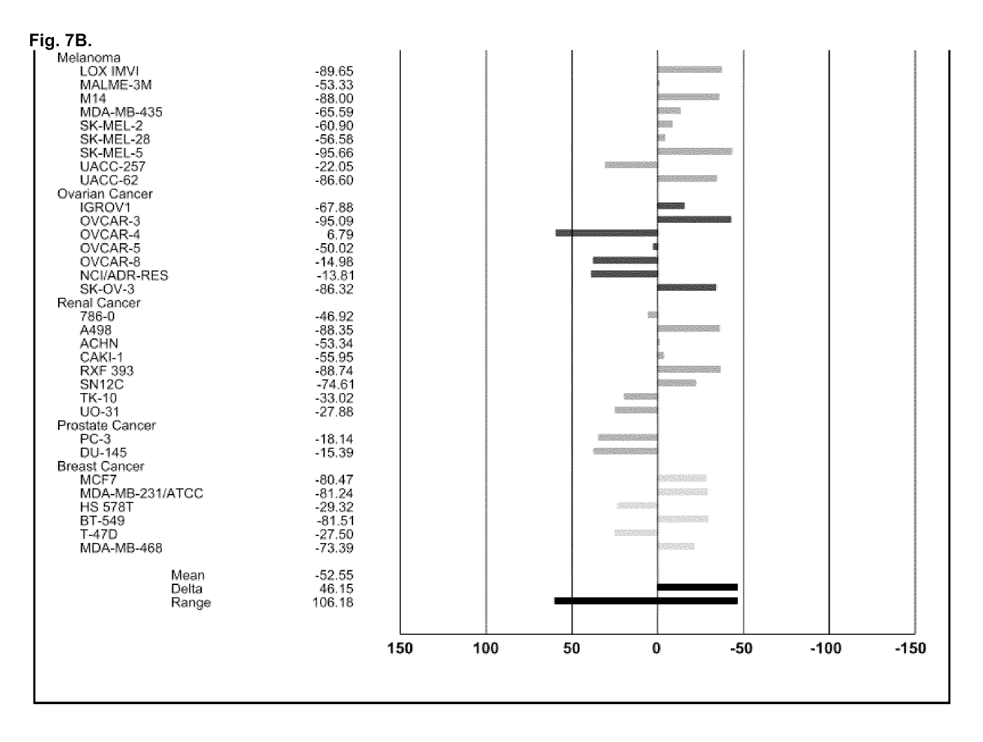

Single dose assay (at 10 μM) showed that thailandepsin A and B are consistently inhibitory to most but not all 60 cancer cell lines, with 43.32% (mean growth percent) or more inhibition of the growth of 28 cell lines by thailandepsin A (6) (FIG. 6), and 52.55% (mean growth percent) or more inhibition of the growth of 30 cell lines by thailandepsin B (7) (FIG. 7). The data suggests that cancer types including colon cancer, melanoma and ovarian cancer are particularly sensitive and susceptible to these compounds.

Five-Dose Assay for Thailandepsins A and B

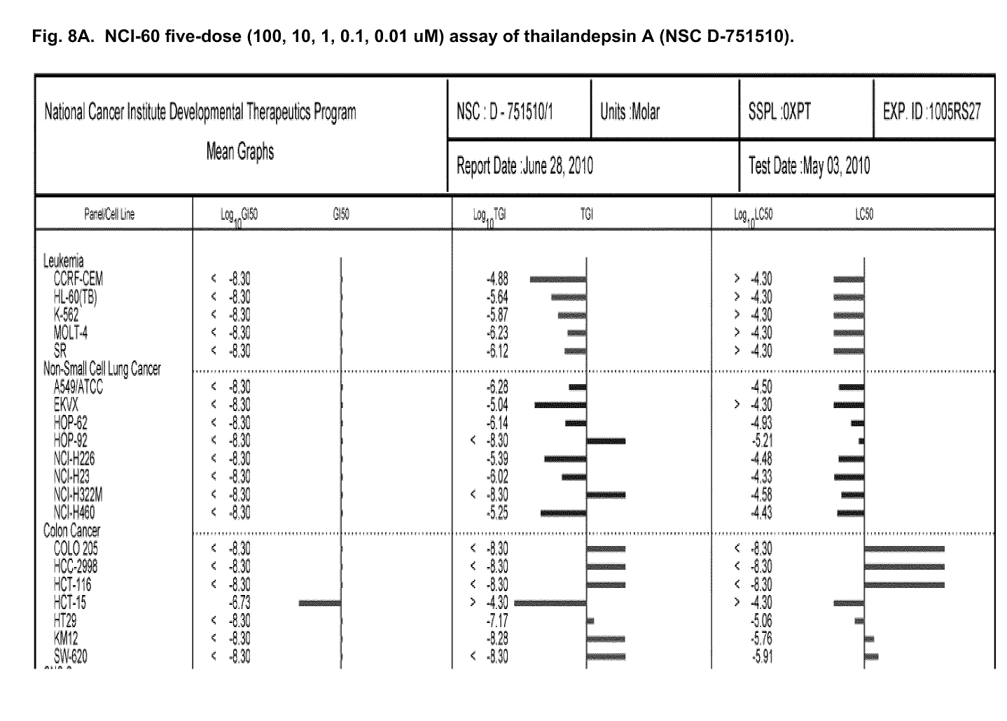

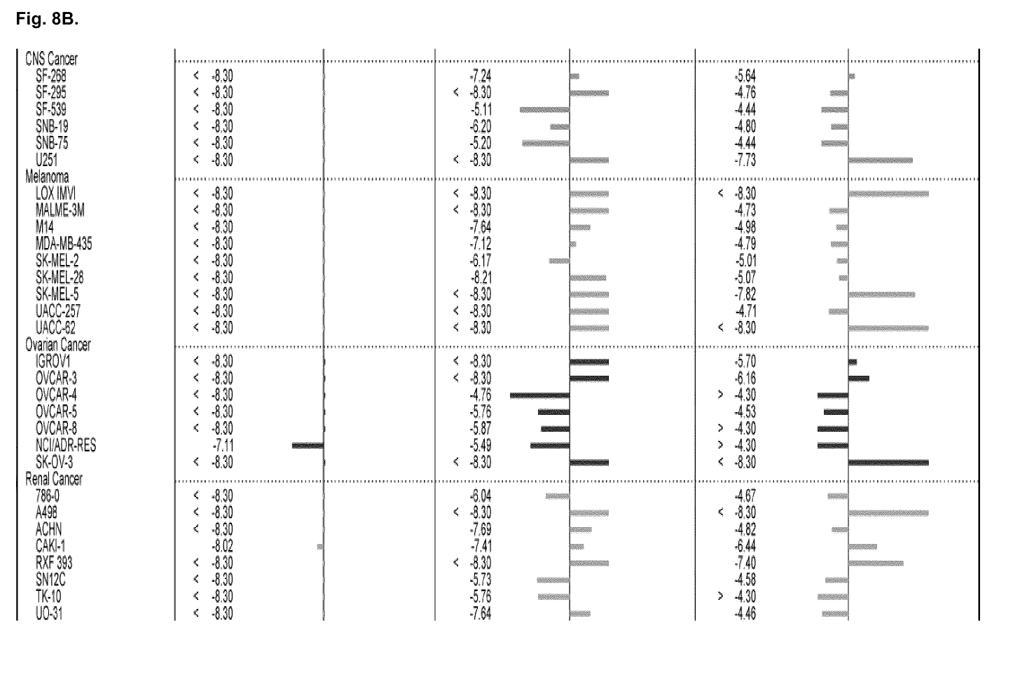

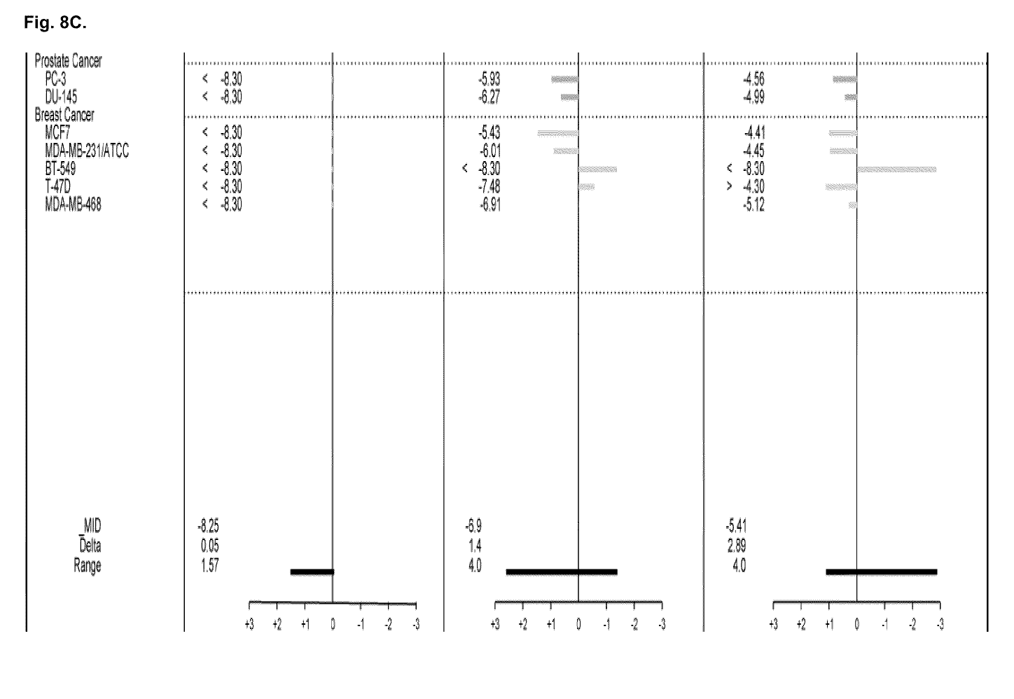

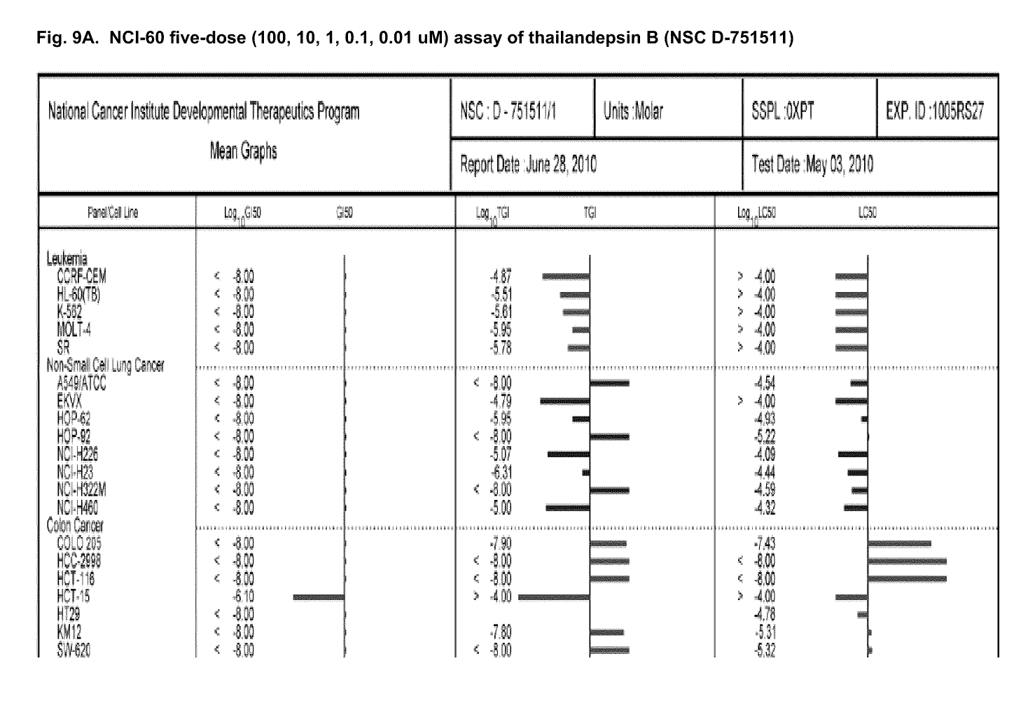

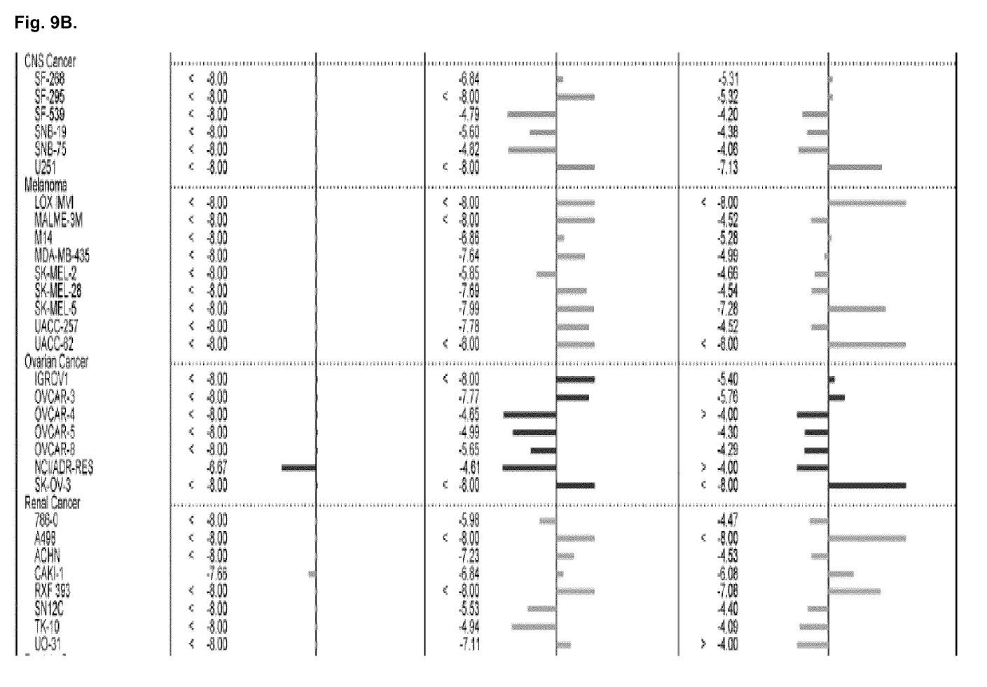

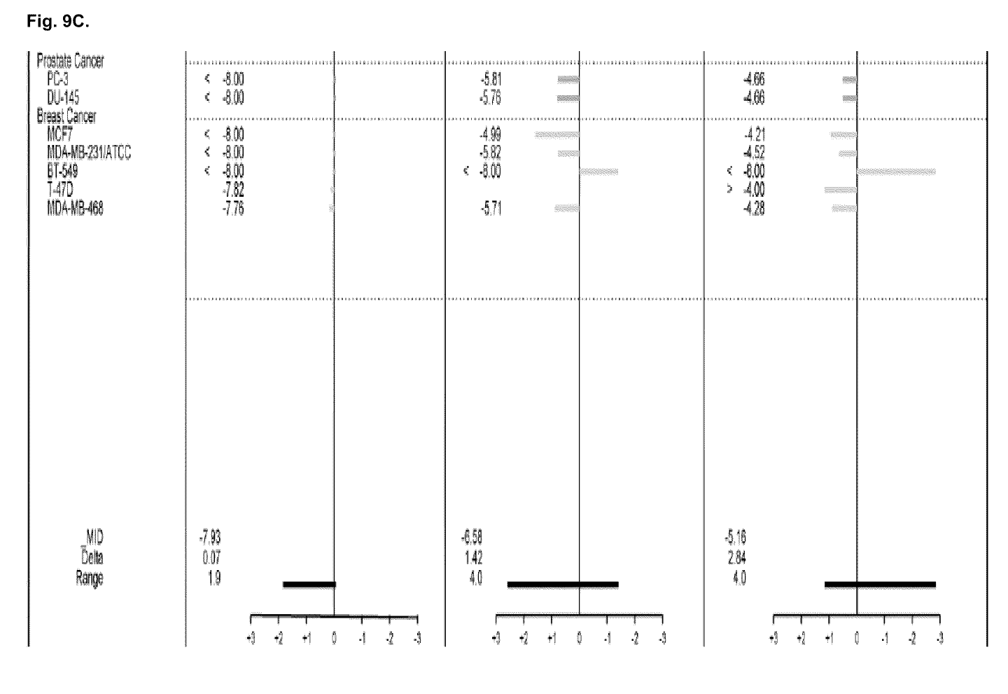

Further evaluation of the inhibitory effect of thailandepsins A and B against various cancer cell lines was made in the NCI-60 five-dose assay (100, 10, 1, 0.1 and 0.01 μM). The results of the assays are provided in FIG. 8 and FIG. 9. The data suggests that: (i) thailandepsin A was inhibitory to all but three cancer cell lines (HCT-15 colon cancer, NCI/ADR-RES ovarian cancer and CAKI-1 renal cancer) at a median concentration of 5.6 nM (10^{-8.25} M; GI50) or lower, while thailandepsin B achieved the same inhibitory effect at a median concentration of 11.7 nM (10^{-7.93} M; GI50) or lower; (ii) thailandepsin A totally inhibited the growth of 29 cancer cell lines at a median concentration of 125.9 nM (10^{-6.9} M; TGI50) or lower, while thailandepsin B totally inhibited the growth of 28 cancer cell lines at a median concentration of 256.4 nM (10^{-6.58} M; TGI50) or lower; (iii) thailandepsin A completely killed 17 cancer cell lines at a median concentration of 3.89 μM (10^{-5.41} M; LC50) or lower, while thailandepsin B completely killed 20 cancer cell lines at a median concentration of 6.92 μM (10^{5.16} M; LC50) or lower; (iv) all parameters indicates that thailandepsin A is twice as potent as thailandepsin B under the conditions of this screening assay; (v) both the five- and single-dose assays suggest that the most susceptible types of cancer to thailandepsins are colon cancer, melanoma, ovarian cancer and renal cancer; and (vi) one CNS cancer cell line (U251) and one breast cancer cell line (BT-549) appear to be very sensitive to both thailandepsin A and B.

Thus, the exemplary data for thailandepsins A and B demonstrate that compounds of Formula I can inhibit HDAC and also have anticancer activity.

We claim:

1. A compound of Formula I: 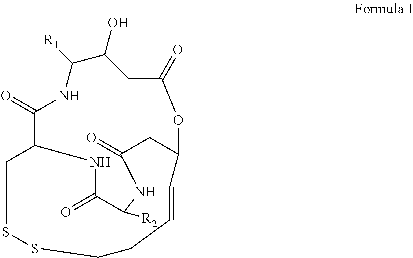
wherein
R₁ and R₂ are independently selected from an amino acid side chain, or derivative thereof, with the proviso that when R₁ is CH(CH₃)₂ or CH(CH₃)CH₂CH₃, R₂ is not CH₃;
or a reduced form of the compound, a dehydration product of the compound, or a salt thereof.

2. A compound of Formula I: 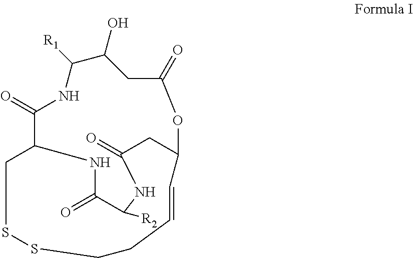
wherein
R₁ is selected from any amino acid side chain, or derivative thereof; and
R₂ is selected from CH₂CH(CH₃)₂, CH(CH₃)CH₂CH₃, (CH₂)₃CH₃, (CH₂)₂SCH₃, (CH₂)₂CONH₂, (CH₂)₃NH₂, (CH₂)₂CO₂, (CH₂)₄NH₃, (CH₂)₃NHC(NH₂)NH₂⁺, (CH₂)₂SeCH₃, 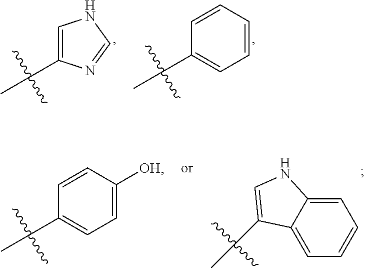
or a reduced form of the compound, a dehydration product of the compound, or a salt thereof.

3. The compound of claim 1, wherein R₂ is CH(CH₃)₂, CH₂SH, CH₂CH(CH₃)₂, CH(CH₃)CH₂CH₃, (CH₂)₃CH₃, (CH₂)₂SCH₃, or CH(OH)CH₃.

4. The compound of claim 1, wherein R₂ is (CH₂)₂SCH₃ or (CH₂)₃CH₃.

5. A pharmaceutical composition comprising the compound of claim 1 and a pharmaceutically acceptable carrier.

6. A method of inhibiting histone deacetylase in a subject in need thereof comprising administering to the subject the compound, or a pharmaceutically acceptable salt thereof, according to claim 1 in an amount effective to inhibit histone deacetylase activity.

7. A method of inhibiting histone deacetylase in a cell, comprising contacting the cell with a compound, or salt thereof, according to claim 1 in an amount effective to inhibit histone deacetylase activity.

8. A method of treating a disease associated with increased histone deacetylase activity in a subject in need thereof comprising administering a compound, or a pharmaceutically acceptable salt thereof, according to claim 1 in an amount effective to treat the disease.

9. A method of increasing histone deacetylase-controlled gene expression in a cell comprising contacting a cell that includes a gene that has its expression controlled by histone deacetylase with the compound of claim 1, or a salt thereof, in an amount effective to increase expression of the gene.

10. A method of inhibiting growth of a cancer cell comprising contacting the cancer cell with an effective amount of a compound, or salt thereof, of Formula I: 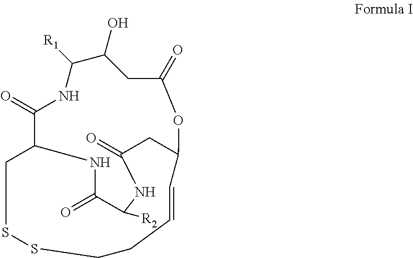
wherein
R₁ and R₂ are independently selected from an amino acid side chain, or derivative thereof, with the proviso that when R₁ is CH(CH₃)₂ or CH(CH₃)CH₂CH₃, R₂ is not CH₃;
or a reduced form or a dehydration product of the compound.

11. The method of claim 10, wherein
R₁ is selected from any amino acid side chain, or derivative thereof; and
R₂ is selected from CH₂CH(CH₃)₂, CH(CH₃)CH₂CH₃, (CH₂)₃CH₃, (CH₂)₂SCH₃, (CH₂)₂CONH₂, (CH₂)₃NH₂, (CH₂)₂CO₂, (CH₂)₄NH₃, (CH₂)₃NHC(NH₂)NH₂⁺, (CH₂)₂SeCH₃,

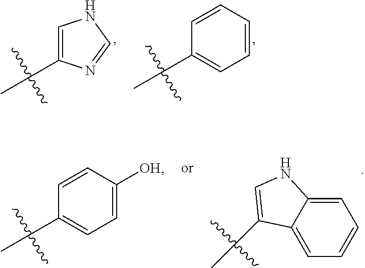

12. A method of treating cancer in a subject in need of such treatment, the method comprising administering to the subject a therapeutically effective amount of the compound of claim 1.

13. The method of claim 12, wherein
R₁ is selected from any amino acid side chain, or derivative thereof; and
R₂ is selected from CH₂CH(CH₃)₂, CH(CH₃)CH₂CH₃, (CH₂)₃CH₃, (CH₂)₂SCH₃, (CH₂)₂CONH₂, (CH₂)₃NH₂, (CH₂)₂CO₂, (CH₂)₄NH₃, (CH₂)₃NHC(NH₂)NH₂⁺, (CH₂)₂SeCH₃,

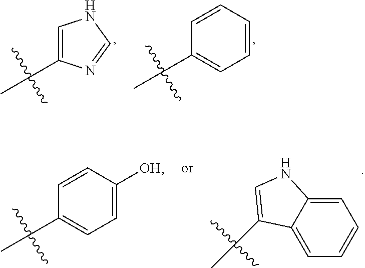

14. The method of claim 12, wherein the therapeutically effective amount is an amount sufficient to inhibit the proliferation of the cancer.

15. The method of claim 12, wherein the therapeutically effective amount is an amount sufficient to slow the progression of the cancer.

16. The method of claim 12, wherein the therapeutically effective amount is an amount sufficient to reduce the number of cancer cells in the subject.

17. The method of claim 12, wherein the cancer is a carcinoma, an adenoma, a melanoma, a sarcoma, a lymphoma, a myeloid leukemia, a lymphatic leukemia, a blastoma, a glioma, an astrocytoma, a mesothelioma, or a germ cell tumor.

18. The method of claim 17, wherein the cancer comprises cancerous cells of the colon, rectum, cervix, skin, eye, epithelium, muscle, kidney/renal, liver/hepatocellular, lymph, bone, blood/hematopoeitic, ovary, prostate, lung, brain/central nervous system, stomach, gastrointestinal, bladder, endometrial/uterine, thyroid, testicle, or breast.

19. The method of claim 18, wherein the cancer is ovarian cancer, renal cancer, colon cancer, melanoma, brain/central nervous system cancer, or breast cancer.

20. The method of claim 12, wherein the method further comprises administering a therapeutically effective amount of an additional anti-cancer agent.
